(19) United States
(12) Patent Application Publication
(10) Pub. No.: US 2004/0049024 A1
(43) Pub. Date: Mar. 11, 2004

(54) Compositions and methods related to the minn1 tumor suppressor gene and protein

(76) Inventors: Geoff Clark, Gaitersburg, MD (US); Michele Vos, Boyds, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/433,836

(22) PCT Filed: Dec. 7, 2001
(86) PCT No.: PCT/US01/48514

(51) Int. Cl. C12Q 1/68; C07H 21/04; C12P 21/02; C12N 5/06; C07K 14/47; C07K 16/18

(52) U.S. Cl. 536/23.5; 530/350; 530/388.1; 435/6; 435/69.1; 435/320.1; 435/325

(57) ABSTRACT

The present invention relates to Ras signalling effector proteins, tumor suppressors and apoptosis. Specifically, the present invention relates to the Ras effector and tumor suppressor gene and protein Minn1 and the regulation/induction of apoptosis. The invention provides compositions and methods for the treatment of cancer, and also relates to the analysis of Minn1 gene structure, transcription and expression.

This application claims priority benefit to U.S. Provisional Patent Application No. 60/251,971, filed Dec. 7, 2000.

This invention was made during the course of work supported by the United States Government, under the National Cancer Institute. As such, the United States Government may have certain rights in this invention.

### FIELD OF THE INVENTION

The present invention relates to Ras signalling effector proteins, tumor suppressors and apoptosis. Specifically, the present invention relates to the Ras effector and tumor suppressor gene and protein Minn1 and the regulation/induction of apoptosis. The invention provides compositions and methods for the treatment of cancer, and also relates to the analysis of Minn1 gene structure, transcription and expression.

### BACKGROUND OF THE INVENTION

Ras-family proteins, also called “small GTP-binding proteins” are utilized by all eukaryotes to transduce extracellular signals which regulate basic cellular functions. These pathways transduce diverse physiological signals in multiple tissues and stages of development.

In the best studied Ras-mediated signal transduction pathways, Ras (also known as “p21”) is activated by receptor tyrosine kinases (RTK), which are located at the cell membrane. There exists a wide variety of RTK proteins, which receive and transmit extracellular signals, which in turn activate Ras proteins. Activated Ras proteins then, in turn, activate other signalling proteins resulting in highly regulated and specific signalling cascades (Katz and McCormick, Curr. Opin. Genet. Dev., 7:75-79 [1997]; Campbell et al., Oncogene 17:1395-1413 [1998]; and Malumbres and Pellicer, Front Biosci 3:d887-d912 [1998]). The downstream components of many of these signalling cascades remain unidentified.

Activated Ras proteins mediate a broad range of biological effects, many of which are associated with enhanced growth and transformation. These effects include reduced growth factor dependence (Andrejauskas and Moroni, EMBO J., 8:2575-2581 [1989]), the induction of DNA synthesis (Mulcahy et al., Nature 313:241-243 [1985]), loss of contact inhibition (Huber and Cordingley, Oncogene 3:245-256 [1988]), inhibition of terminal differentiation (Yuspa et al., Nature 314:459-462 [1985]), resistance to apoptosis (Kauffmann-Zeh et al., Nature 385:544-548 [1997]), enhanced motility (Trahey et al., Mol. Cell Biol., 7:541-544 [1987]), metastasis/invasion (Ochieng et al., Invasion Metastasis 11:38-47 [1991]; and Takiguchi et al., Clin. Exp. Metastasis 10:351-360 [1992]) and tumorigenic transformation (Barbacid, Annu. Rev. Biochem., 56:779-827 [1987]; and Lowy and Willumsen, Annu. Rev. Biochem., 62:851-891 [1993]).

The signaling activity of the Ras protein is modulated by its bound guanine nucleotide. Ras protein which binds the trinucleotide GTP is in an active conformation, while Ras protein which binds the dinucleotide GDP is inactive (McCormick, Nature 363:15 [1993]; and Marshall, Curr. Opin. Genet. Dev., 4(1):82-92 [1994]). Following the binding of GTP, intrinsic GTPase activity within the Ras protein hydrolyses the terminal phosphate of the GTP to yield GDP, which is then exchanged for another molecule of GTP. The GTPase and nucleotide exchange activities intrinsic to Ras are augmented by other regulatory proteins. The ancillary proteins Ras-GTPase activating protein (GAP) and guanine nucleotide exchange factor (GNEF) also contribute to the potency of Ras signaling, and are important modulators of the Ras-signal. Some human genetic diseases have been attributed to mutations in genes encoding these proteins.

Mammalian cells are known to have at least three Ras proteins, namely, H-Ras, K-Ras and N-Ras. These Ras proteins, although sharing a highly conserved structure, have been shown to serve different functions in a cell. In addition, there are families of more distantly related small GTP-binding proteins, including Rac, Rho, CDC42, TC21, Rit, Ral, and Rap (Campbell et al., Oncogene 17:1395-1413 [1998]; and Malumbres and Pellicer, Front Biosci 3:d887-d912 [1998])

Despite the fact that this model for Ras-dependent signal transduction has been extensively studied for a number of years, little is known how so many extracellular signals are able to use the finite number of RTK and Ras proteins in a cell. Indeed, for most Ras signalling pathways, little is known of the events which occur following Ras activation, and the proteins involved in the events following Ras activation remain largely unidentified.

Ras Signaling in Cancer

Activated Ras proteins play a key role in the development of human cancers. Mutations in Ras are observed in approximately one third of all tumors (Bos, Cancer Res 49:4682-4689 [1989]; and Clark and Der, in GTPases in Biology [eds. Dickey and Birmbauer], Springer-Verlag London Ltd., pp. 259-287 [1993]). Indeed, the frequency of Ras mutation approaches 100% in some types of tumors (e.g., pancreatic adenocarcinoma). These mutated Ras proteins demonstrate decreased inherent GTPase activity, and are resistant to the action of GTPase-activating proteins (GAPs). Thus, these mutations are activating mutations resulting in the Ras protein being locked in an active conformation, leading ultimately to inappropriate cell proliferation signaling. Furthermore, activated forms of the Ras protein are useful in the induction of tumors, thereby providing direct evidence for Ras involvement in malignant cell transformation and tumorigenesis. Moreover, deletion of the activated Ras gene from tumor cell lines impairs their tumorigenicity (Paterson et al., Cell 51:803-812 [1987]; and Shirasawa et al., Science 260:85-88 [1993]).

Apoptosis

Apoptosis (also referred to as “programmed cell death”) is a highly regulated cellular mechanism which controls cell suicide. The apoptosis pathway is activated in order to remove excess, damaged, abnormal, infected or potentially harmful cells from the body. The removal of such cells is a normal event during development and homeostasis of multicellular organisms. The initiation of apoptosis is controlled by signalling pathways leading ultimately to the activation of caspase enzymes and programmed cell destruction. Apoptosis is initiated by a variety of intracellular or extracellular stimuli, and a large number of proteins involved in apoptosis are known. For example, apoptosis can be initiated by an extracellular “death signal” known as the Fas ligand (also termed FasL or CD95L) which activates a specific receptor, termed Fas (also known as Fas receptor, CD95 or APO-1) at the extracellular surface of the plasma membrane, leading to the sequential activation of a cascade of signaling proteins, ultimately resulting in apoptosis.

There is a need in the art to identify genes and proteins involved in the regulation of cell proliferation and apoptosis. There is a need for improved understanding of Ras-family protein signalling in order to better understand the molecular mechanisms of cancer. There is also a need in the art for compositions and methods which have the ability to induce apoptosis and control unregulated or harmful cell survival or proliferation. Such compositions and methods have therapeutic value. For example, such compositions and methods find use in the eradication of tumors.

### SUMMARY OF THE INVENTION

The present invention provides a Ras-effector gene and protein with tumor suppressor activity. It is contemplated that this gene and protein, called “Minn1,” will find use in the treatment of tumors, and most preferably, for the treatment of tumors that show deletion or mutation of the endogenous Minn1 gene and/or reduced expression of the Minn1 transcript or protein.

In one embodiment, the present invention provides isolated nucleic acids encoding the polypeptide set forth in SEQ ID NO:2 (i.e., the Minn1 protein). In a preferred embodiment, this isolated nucleic acid comprises the nucleotide sequence set forth in SEQ ID NO:1. In another embodiment, the present invention provides an isolated Minn1 polypeptide having the amino acid sequence of SEQ ID NO:2.

In other embodiments, the present invention provides compositions comprising a nucleic acid encoding the Minn1 polypeptide (i.e., SEQ ID NO:2). In one embodiment, the invention provides recombinant DNA vectors comprising a nucleic acid encoding the Minn1 polypeptide. In related embodiments, the recombinant DNA vector is an expression vector. In other embodiments, a host cell comprises the recombinant DNA vector, where the host cell is either prokaryotic (i.e., a bacterial cell) or eukaryotic (e.g., a mammalian cell).

The present invention also provides purified antibodies directed against the Minn1 polypeptide, or any portion of the Minn1 polypeptide. In some embodiments, the antibody is monoclonal, while in other embodiments, the antibody is polyclonal. In a related embodiment, the invention provides compositions comprising anti-Minn1 antibody. In further embodiments, the present invention provides antibodies that are specifically directed against an isoform of Minn1. For example, in some embodiments, the antibodies are directed against Minn1A, while in other embodiments, the antibodies are directed agains Minn1C. These anti-isoform antibodies find use alone, as well as in combination in the methods of the present invention.

The present invention also provides methods for treating a subject, comprising the steps of: (a) providing a subject, a recombinant vector encoding the Minn1 polypeptide, a target within the subject, and a means of delivery of the vector to the target within the subject, and (b) delivering the vector to the target within the subject using the means of delivery. In one preferred embodiment, the subject is a human. In another preferred embodiment, the subject displays a solid tumor, and the target of the method is the solid tumor.

In other embodiments of this method, the cells which make up the solid tumor have at least one mutation in at least one Ras-family gene, where the mutation results in increased Ras signalling activity. In another embodiment of the method, the cells making up the solid tumor show reduced levels of either Minn1 transcript and/or Minn1 polypeptide relative to non-tumor tissue of like origin. In a particularly preferred embodiment of the method, the cells which make up the solid tumor have at least one mutation in at least one Ras-family gene, where the mutation results in increased Ras signalling activity in addition to showing reduced levels of either Minn1 transcript and/or Minn1 polypeptide relative to non-tumor tissue of like origin. In one embodiment, the solid tumor is an ovarian tumor.

In other embodiments of this method, the delivery of the nucleic acid encoding the Minn1 protein uses either administration of a liposome-DNA complex or infection with a recombinant virus. In preferred embodiments, the recombinant virus uses a suitable operably-linked promoter sequence to promote expression of the Minn1 polypeptide, and the recombinant virus comprises viral sequences derived from adenovirus, adeno-associated virus, retrovirus, herpes virus, vaccinia virus or Moloney virus. In other preferred embodiments, the means of delivery is selected from local surgical delivery, implantation, and localized injection.

The present invention also provides methods for detecting a Minn1 polypeptide in a sample, comprising (a) providing a sample and an antibody directed against a Minn1 polypeptide, (b) contacting said sample with the antibody under conditions such that the antibody specifically binds to Minn1 polypeptide in the sample to form an antigen-antibody complex, and (c) detecting the antigen-antibody complex. In one embodiment, the sample is from a human subject. In another embodiment, the sample is tumor tissue. In some embodiments, the method comprises Western immunoblotting, while in other embodiments, the method comprises an enzyme-linked immunosorbent assay (ELISA). In some embodiments, the ELISA is selected from the group consisting of direct ELISA, indirect ELISA, direct sandwich ELISA, indirect sandwich ELISA, and competitive ELISA.

The present invention also provides methods for detecting a Minn1 transcript in a sample. This method comprises (a) providing a sample, where the sample is total cellular RNA or polyA RNA, a nucleic acid probe having complementarity to at least a portion of the nucleotide sequence encoding the Minn1 protein, a means of detecting a hybridization complex comprising the probe, (b) combining the nucleic acid probe and the sample under conditions suitable for the formation of a hybridization complex between the probe and the Minn1 transcript, and (c) detecting the hybridization complex. In one embodiment, the sample is from a human subject. In a preferred embodiment, the sample is derived from tumor tissue. In a most preferred embodiment of this method, the method comprises Northern blotting.

The present invention further provides additional methods for detecting Minn1 transcript in a sample. In a most preferred embodiment, this method is a reverse transcriptase polymerase chain reaction (RT-PCR) method. This method comprises (a) providing a sample, where the sample comprises either total cellular RNA or polyA RNA; a reverse transcriptase; PCR primers having complementarity to the nucleotide sequence of SEQ ID NO:1; a DNA-dependent DNA polymerase; and PCR amplification reagents; and (b) reverse transcribing the RNA in the sample to form a double stranded DNA template, (c) annealing the primers to the template, (d) extending the primers with reiterated DNA synthesis under conditions such that the template is amplified to produce an amplified PCR product; and (e) detecting the amplified PCR product. In one embodiment, the sample is from a human subject. In a preferred embodiment, the sample is derived from tumor tissue.

The present invention also provides methods for detecting deletion mutations in a Minn1 genomic locus using PCR technology. These methods comprise (a) providing a first sample of genomic DNA from tumor tissue, a second sample of genomic DNA from a non-tumorigenic tissue, PCR primers, a DNA-dependent DNA polymerase, PCR amplification reagents, and (b) annealing the primers to the genomic DNA, (c) extending the primers with reiterated DNA synthesis to produce an amplified PCR product, (d) detecting the amplified PCR products, and (e) comparing the amplified products from the tumor and non-tumor samples. In a preferred embodiment, the tumor and non-tumor samples are from a human subject. In an alternative embodiment, the DNA-dependent DNA polymerase is a thermostable DNA polymerase.

The present invention also provides methods for detecting a Minn1 polypeptide in an array of tissue samples, comprising the steps of: providing tissue array comprising at least two tissue samples, and an antibody directed against a Minn1 polypeptide; contacting the tissue samples with the antibody under conditions such that the antibody specifically binds to the Minn1 polypeptide in the tissue samples to form an antigen-antibody complex; and detecting the antigen-antibody complex. In some preferred embodiments, at least one of the tissue samples is from a human subject. In other preferred embodiments, the comprises tumor tissue. In still further embodiments, the method comprises an immunohistochemical testing assay. In yet further embodiments, the tissue array comprises more than 100 tissue samples. In some particularly preferred embodiments, the tissue array comprises tissue samples from normal and tumor tissues (Le., negative and positive control samples). In still further preferred embodiments, the step of determining the cell type in the tissue sample that exhibits the antigen-antibody complex is also conducted. Thus, the present invention provides means to determine the cell types within a test tissue sample that express differing levels of Minn1. This provides additional information to the clinician regarding the disease status of the patient, as well as an indication of treatment options and prognosis.

### DESCRIPTION OF THE FIGURES

FIG. 1 shows the nucleotide sequence of the human Minn1 open reading frame of the present invention (SEQ ID NO:1).

FIG. 2 shows the amino acid sequence of the Minn1 protein of the present invention (SEQ ID NO:2).

FIG. 3 shows a Western immunoblot using an anti-Ras antibody following an in vitro protein binding assay using GTP-bound Ras, GDP-bound Ras, and the Minn1 protein.

FIG. 4 shows a Western immunoblot using an anti-FLAG antibody following an in vivo protein binding assay using FLAG-tagged Minn1 protein and an HA-tagged H-Ras protein following co-transfection.

FIG. 5 shows a Northern blot using RNA from human tissues and a Minn1 cDNA probe.

FIG. 6 shows a Northern blot using total RNA from normal and transformed ovarian cell lines and a Minn1 cDNA probe.

FIG. 7, Panel A, shows colony formation following transfection and stable selection of N1H-3T3 cells with either a Minn1 expression vector (bottom) or an empty control vector (top). FIG. 7, Panel B, shows phase contrast microscopic images of 293-T cells transiently transfected with either a Minn1 expression vector (bottom) or an empty control vector (top).

FIG. 8 shows phase contrast microscopic images of 293-T cells transiently co-transfected with either a Minn1 expression vector (top row) or a corresponding empty control vector (bottom row), in addition to expression vectors encoding activated H-Ras, dominant negative H-Ras, Ras carrying an effector domain mutation, or a corresponding control vector.

FIG. 9 shows phase contrast microscopic images of 293-T cells transiently transfected with expression vectors encoding Minn1, Fas or an empty control vector, and shows the response of these cells to the caspase inhibitor Z-VAD-FMK (bottom row) and carrier alone (top row).

FIG. 10 provides a Western blot showing differential expression of Minn1A and Minn1C in ovarian tumor cell lines.

FIG. 11 provides a Western blot of lung cancer cell lines tested with antibodies directed against Minn1. As indicated in this Figure, Minn1C expression is frequently lost in lung cancer cell lines.

FIG. 12 provides a Western blot of breast tumor cell lines tested with antibodies directed against Minn1. As indicated in this Figure, Minn1C expression is frequently lost in breast tumor cell lines.

FIG. 13 provides a Western blot showing that endogenous Ras and Minn1 interact in vivo.

### GENERAL DESCRIPTION OF THE INVENTION

The present invention relates to a novel Ras effector gene having tumor suppressor activity. Surprisingly, the protein encoded by this gene has the ability to induce apoptosis in the presence of activated Ras, and is dependent on Ras activity for apoptosis-inducing activity. Specifically, the present invention provides the human Minn1 gene and the protein encoded by this gene. In addition, the present invention also provides recombinant vectors comprising the gene, host cells comprising the vectors and antibodies specific for the Minn1 protein.

The compositions of the present invention find use in the treatment of cancer, where the Minn1 gene is delivered to the cancer cells of a subject by gene therapy methods. Furthermore, the present invention provides compositions and methods for the detection of the Minn1 gene and protein.

An understanding of the mechanism of Minn1 activity is not required in order to make or use the present invention. Furthermore, is it not intended that the present invention be limited to any particular proposed mechanism of action.

Definitions

To facilitate understanding of the invention, a number of terms are defined and discussed below.

The terms “nucleic acid,” “nucleic acid sequence,” “nucleotide sequence,” “oligonucleotide,” “polynucleotide” or “nucleic acid molecule” as used herein refer to an oligonucleotide or polynucleotide, and fragments or portions thereof, and to DNA or RNA of genomic or synthetic origin which can be single- or double-stranded, and represent the sense or antisense strand. Similarly, “amino acid sequence” as used herein refers to the primary sequence of amino acids in a peptide, polypeptide or protein.

The term “nucleotide” as used herein refers to any nucleotide that comprises any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “oligonucleotide,” refers to a short length of single-stranded polynucleotide chain. Oligonucleotides are typically less than 100 nucleotides long (e.g., between 15 and 50), however, as used herein, the term is also intended to encompass longer polynucleotide chains. Oligonucleotides are often referred to by their length. For example a 24 residue oligonucleotide is referred to as a “24-mer.” Oligonucleotides can form secondary and tertiary structures by self-hybridizing or by hybridizing to other polynucleotides. Such structures include, but are not limited to, duplexes, hairpins, cruciforms, bends, and triplexes.

As used herein, “recombinant nucleic acid,” “recombinant gene” “recombinant DNA molecule” or similar terms indicate that the nucleotide sequence or arrangement of its parts is not a native configuration, and has been manipulated by molecular biological techniques. The term implies that the DNA molecule is comprised of segments of DNA that have been artificially joined together. Protocols and reagents to manipulate nucleic acids are common and routine in the art (See e.g., Maniatis et al. (eds.), Molecular Cloning: A Laboratory Manual, Cold Spring Harbor Laboratory Press, NY, [1982]; Sambrook et al. (eds.), Molecular Cloning: A Laboratory Manual, Second Edition, Volumes 1-3, Cold Spring Harbor Laboratory Press, NY, [1989]; and Ausubel et al. (eds.), Current Protocols in Molecular Biology, Vol. 1-4, John Wiley & Sons, Inc., New York [1994]).

As used herein, the terms “restriction endonucleases” and “restriction enzymes” refer to bacterial enzymes, each of which cut double-stranded DNA at or near a specific nucleotide sequence.

As used herein, the term “probe” refers to an oligonucleotide (i.e., a sequence of nucleotides), which is often produced from nucleic acid isolated from cells (typically a recombinant nucleic acid), produced synthetically or in vitro, which is capable of hybridizing to a nucleic acid of interest. Probes are useful in the detection, identification and isolation of particular gene sequences. It is contemplated that any probe used in the present invention is capable of being labelled with any “reporter molecule,” so that the probe is detectable. Detection systems include, but are not limited to, the detection of enzymatic activity, fluorescence, radioactivity, and luminescence. It is not intended that the present invention be limited to any particular probe, label or detection system.

As used herein, the terms “complementary” or “complementarity” are used in reference to antiparallel polynucleotides (i.e., a sequence of nucleotides) related by the base-pairing rules. For example, the sequence 5′-AGTTC-3′ is complementary to the sequence 3′-TCAAG-5′. Complementarity can be “partial,” in which only some of the nucleic acids' bases are matched according to the base pairing rules. Or, there can be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology,” as it applies to nucleotide sequences, refers to a degree of complementarity. It is intended that the term encompass partial homology as well as complete homology (i.e., 100% identity). A partially complementary sequence is one that at least partially inhibits a completely complementary sequence from hybridizing to a target nucleic acid, and is referred to using the functional term “substantially homologous.” The inhibition of hybridization of the completely complementary sequence to the target sequence can be examined using a hybridization assay (Southern or Northern blot, solution hybridization and the like) under conditions of low stringency. A substantially homologous sequence or probe will compete for and inhibit the binding (i.e., the hybridization) of a completely homologous sequence to a target sequence under conditions of low stringency. This is not to say that conditions of low stringency are such that non-specific binding is permitted; low stringency conditions require that the binding of two sequences to one another be a specific (i.e., selective) interaction. The absence of non-specific binding can be tested by the use of a second target which lacks even a partial degree of complementarity (e.g., less than about 30% identity); in the absence of non-specific binding, the probe will not hybridize to the second non-complementary target.

The term “hybridization” as used herein includes “any process by which a strand of nucleic acid joins with a complementary strand through base pairing” (Coombs, Dictionary of Biotechnology, Stockton Press, New York N.Y. [1994]. Hybridization can be demonstrated using a variety of hybridization assays (Southern blot, Northern Blot, slot blot, phage plaque hybridization, and other techniques). These protocols are common in the art (See e.g., Sambrook et al. (eds.), Molecular Cloning: A Laboratory Manual, Second Edition, Volumes 1-3, Cold Spring Harbor Laboratory Press, NY, [1989]; Ausubel et al. (eds.), Current Protocols in Molecular Biology, Vol. 1-4, John Wiley & Sons, Inc., New York [1994]).

Hybridization may occur between two antiparallel nucleic acids which may or may not have 100% complementarity. Two nucleic acids which contain 100% antiparallel complementarity will show strong hybridization. Two antiparallel nucleic acids which contain no antiparallel complementarity (generally considered to be less than 30%) will not hybridize. Two nucleic acids which contain between 31-99% complementarity will show an intermediate level of hybridization. A single molecule that contains pairing of complementary nucleic acids within its structure is said to be “self-hybridized.”

As used herein, the term “stringency” is used in reference to the conditions of temperature, ionic strength, and the presence of other compounds such as organic solvents, under which nucleic acids hybridize. “Low or weak stringency” conditions are reaction conditions which favor the complementary base pairing and annealing of two nucleic acids. “High stringency” conditions are those conditions which are less optimal for complementary base pairing and annealing. The art knows well that numerous variables affect the strength of hybridization, including the length and nature of the probe and target (DNA, RNA, base composition, present in solution or immobilized, the degree of complementary between the nucleic acids, the Tₘ of the formed hybrid, and the G:C ratio within the nucleic acids). Conditions can be manipulated to define low or high stringency conditions: factors such as the concentration of salts and other components in the hybridization solution (e.g., the presence or absence of formamide, dextran sulfate, polyethylene glycol) as well as temperature of the hybridization and/or wash steps. Conditions of “low” or “high” stringency are specific for the particular hybridization technique used.

During hybridization of two nucleic acids under high stringency conditions, complementary base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less. As used herein, two nucleic acids which are able to hybridize under high stringency conditions are considered “substantially homologous.”

The art knows well that numerous equivalent conditions can be employed to comprise either low or high stringency nucleic acid hybridization conditions; factors such as the length and composition of the probe (DNA, RNA, base sequence) and composition of the target (DNA, RNA, base sequence, present in solution or immobilized, etc.) and the concentration of the salts and other components (e.g., the presence or absence of formamide, dextran sulfate, polyethylene glycol) are considered in selecting suitable hybridization conditions. The hybridization solution can be varied to generate conditions for either low or high stringency hybridization. Conditions which constitute high or low stringency are common to one familiar with the art, and are described in numerous sources (e.g., Anderson and Young, Quantitative Filter Hybridization, in Nucleic Acid Hybridization [1985] and Ausubel et al. (eds.), Current Protocols in Molecular Biology, Vol. 1-4, John Wiley & Sons, Inc., New York [1994]).

“Stringency” typically occurs in a range from about Tₘ-5° C. (i.e., 5° C. below the Tₘ of the probe) to about 20° C. to 25° C. below Tₘ. As will be understood by those of skill in the art, a stringent hybridization can be used to identify or detect identical polynucleotide sequences or to identify or detect similar or related polynucleotide sequences.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value can be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations which take structural as well as sequence characteristics into account for the calculation of Tₘ.

Whether sequences are “substantially homologous” can be verified using hybridization competition assays. For example, a “substantially homologous” nucleotide sequence is one that at least partially inhibits a completely complementary probe sequence from hybridizing to a target nucleic acid under conditions of low stringency. This is not to say that conditions of low stringency are such that non-specific binding is permitted; low stringency conditions require that the binding of two sequences to one another be a specific (i.e., selective) interaction. The absence of non-specific binding can be verified by the use of a second target that lacks even a partial degree of complementarity (e.g., less than about 30% identity); in the absence of non-specific binding the probe will not hybridize to the second non-complementary target. When used in reference to a double-stranded nucleic acid sequence such as a cDNA or genomic clone, the term “substantially homologous” refers to any probe that is capable of hybridizing to either or both strands of the double-stranded nucleic acid sequence under conditions of high stringency.

A gene may produce multiple RNA species that are generated by differential splicing of the primary RNA transcript. cDNAs that are splice variants of the same gene contain regions of nucleotide sequence identity (100% homology), representing the presence of the same exon or portion of the same exon on both cDNAs, and regions of non-identity. The two cDNAs contain regions of nucleotide sequence that will hybridize to a probe derived from the entire gene or portions of the gene containing sequences found on both cDNAs. As used herein, the two splice variants are therefore substantially homologous to such a probe and to each other.

As used herein the term “hybridization complex” refers to a complex formed between two nucleic acid sequences by virtue of the formation of hydrogen bonds between complementary G and C bases and between complementary A and T bases; these hydrogen bonds can be further stabilized by base stacking interactions. The two complementary nucleic acid sequences hydrogen bond in an antiparallel configuration. A hybridization complex can be formed in solution or between one nucleic acid sequence present in solution and another nucleic acid sequence immobilized to a solid support (e.g., a nylon membrane or a nitrocellulose filter as employed in Southern and Northern blotting, dot blotting or a glass slide as employed in in situ hybridization, including FISH [i.e., fluorescent in situ hybridization]).

As used herein, the term “antisense” is used in reference to any nucleic acid which is antiparallel to and complementary to another nucleic acid. The present invention encompasses antisense DNA and RNA produced by any method. For example, in some embodiments, a cDNA or a portion of a cDNA is subcloned into an expression vector containing a promoter which permits transcription either in vitro or in vivo. The cDNA or a portion of the cDNA is subcloned in such a way that it is in the reverse orientation relative to the direction of transcription of the cDNA in its native chromosome. Transcription of this antisense cDNA produces an RNA transcript that is complementary and antiparallel to the native mRNA. In alternative embodiments, the antisense nucleic acid is a synthetically-produced oligonucleotide. The mechanism by which an antisense nucleic acid produces effects in a biological system is unclear. In some embodiments, antisense techniques are used to produce an “artificial knockout” mutant in an animal or animal cell line. The term “antisense strand” is used in reference to the nucleic acid strand that is complementary to the “sense” strand. The designation (−) (i.e., “negative”) is sometimes used in reference to the antisense strand, with the designation (+) (i.e., “positive”) sometimes used in reference to the sense strand.

“Amplification” is defined as the production of additional copies of a nucleic acid sequence and is generally carried out using polymerase chain reaction (PCR) or other technologies well known in the art (e.g., Dieffenbach and Dveksler, PCR Primer, a Laboratory Manual, Cold Spring Harbor Press, Plainview N.Y. [1995]). As used herein, the term “polymerase chain reaction” (“PCR”) refers to the method of K. B. Mullis (U.S. Pat. Nos. 4,683,195, 4,683,202 and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension (DNA synthesis) are typically reiterated many times (i.e., denaturation, annealing and extension constitute one “cycle”; there usually are numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

As used herein, the term “polymerase” refers to any polymerase suitable for use in the amplification of nucleic acids of interest. It is intended that the term encompass such DNA polymerases as Taq DNA polymerase obtained from Thermus aquaticus, although other polymerases, both thermostable and thermolabile, are also encompassed by this definition.

As used herein, the term “primer” refers to an oligonucleotide, typically but not necessarily produced synthetically, that is capable of acting as a point of initiation of nucleic acid synthesis when placed under conditions in which synthesis of a primer extension product that is complementary to a nucleic acid strand is induced, (i.e., in the presence of nucleotides, an inducing agent such as DNA polymerase, and at a suitable temperature and pH). The primer is preferably single stranded for maximum efficiency in amplification, but in alternative embodiments, it is double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “nested primers” refers to primers that anneal to the target sequence in an area that is inside the annealing boundaries used to start PCR. (See, K. B. Mullis, et al., Cold Spring Harbor Symposia, Vol. LI, pp. 263-273 [1986]). Because the nested primers anneal to the target inside the annealing boundaries of the starting primers, the predominant PCR-amplified product of the starting primers is necessarily a longer sequence, than that defined by the annealing boundaries of the nested primers. The PCR-amplified product of the nested primers is an amplified segment of the target sequence that cannot, therefore, anneal with the staring primers. As used herein, the term “amplification reagents” refers to those reagents (deoxyribonucleoside triphosphates, buffer, etc.), needed for amplification except for primers, nucleic acid template and the amplification enzyme.

As used herein, the term “amplification reagents” refers to those reagents (e.g., deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

Using PCR and an appropriate set of primer molecules, it is possible to amplify a single copy of a specific target sequence in genomic DNA, cDNA, mRNA or any other nucleic acid, to a level detectable by several different methodologies (e.g., ethidium bromide visualization; hybridization with a labelled probe; incorporation of biotinylated primers followed by avidin-enzyme conjugate detection; incorporation of ³²P-labeled deoxynucleotide triphosphates, such as dCTP or dATP, into the amplified segment). In particular, the amplified segments created by the PCR process itself are, themselves, efficient templates for subsequent PCR amplifications. Amplified target sequences are useful to obtain segments of DNA (e.g., genes) for insertion into recombinant vectors.

As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

The terms “peptide,” “polypeptide” and “protein” all refer to a primary sequence of amino acids that are joined by covalent “peptide linkages.” In general, a peptide consists of a few amino acids, typically from 2-25 amino acids, and is shorter than a protein. “Polypeptides” encompass both peptides or proteins. As used herein, a recited “amino acid sequence” refers to an amino acid sequence of a naturally occurring protein molecule, a protein produced by recombinant molecular genetic techniques, or a synthetic or naturally occurring peptide, and may refer to a portion of a larger “peptide,” “polypeptide” or “protein,” and is not meant to limit the amino acid sequence to the complete, native amino acid sequence associated with the recited protein molecule.

A “deletion” is defined as a change in either nucleotide or amino acid sequence in which one or more nucleotides or amino acid residues, respectively, are absent. The deletion of an entire gene locus is frequently designated by the symbol “Δ” followed by the gene name.

A “recombinant protein” or “recombinant polypeptide” refers to a protein molecule that is expressed from a recombinant DNA molecule. Use of these terms indicates that the primary amino acid sequence, arrangement of its domains or nucleic acid elements which control its expression are not native, and have been manipulated by molecular biology techniques. As indicated above, techniques to manipulate recombinant proteins are also common and routine in the art.

“Isoforms” refer to families of functionally-related proteins that differ slightly in their amino acid sequences. These protein isoforms arise from the same gene by a process of differential exon usage.

The terms “exogenous” and “heterologous” are sometimes used interchangeably with “recombinant.” An “exogenous nucleic acid,” “exogenous gene” and “exogenous protein” indicate a nucleic acid, gene or protein, respectively, that has come from a source other than its native source, and has been artificially supplied to the biological system. In contrast, the terms “endogenous protein,” “native protein,” “endogenous gene,” and “native gene” refer to a protein or gene that is native to the biological system, species or chromosome under study. A “native” or “endogenous” gene is a gene that does not contain nucleic acid elements encoded by sources other than the chromosome on which it is normally found in nature. An endogenous gene or transcript is encoded by its natural chromosomal locus, and not artificially supplied to the cell.

As used herein the term “portion” when in reference to a protein (as in “a portion of a given protein”) refers to fragments of that protein. In some embodiments, the fragments range in size from four amino acid residues to the entire amino acid sequence minus one amino acid. In other embodiments, the “portion” is further limited to only fragments of the full length protein that retain biological activity. For example, a portion of the Minn1 protein is a fragment of the Minn1 protein that retains the ability to induce apoptosis in a Ras-dependent manner.

A “variant” in regard to amino acid sequences is used to indicate an amino acid sequence that differs by one or more amino acids from another sequence, and additionally where that variant retains the biological activity of the parent molecule. In some embodiments, the variant has “conservative” changes, wherein a substituted amino acid has similar structural or chemical properties (e.g., replacement of leucine with isoleucine). More rarely, a variant has “non-conservative” changes (e.g., replacement of a glycine with a tryptophan). Similar minor variations also include amino acid deletions or insertions (i.e., additions), or both. Guidance in determining which and how many amino acid residues may be substituted, inserted or deleted without abolishing biological or immunological activity is often provided by computer programs well known in the art (e.g., DNAStar). Thus, it is contemplated that this definition encompasses variants of Minn1. In some embodiments, these variants are tested in functional assays (e.g., growth inhibition assays).

The following definitions are the commonly accepted definitions of the terms “identity,” “similarity” and “homology.” Percent identity, as it applies to polypeptides, is a measure of strict amino acid conservation. Percent similarity is a measure of amino acid conservation which incorporates both strictly conserved amino acids, as well as “conservative” amino acid substitutions, where one amino acid is substituted for a different amino acid having similar chemical properties (i.e., a “conservative” substitution). In some embodiments, the term “homology” pertains to either proteins or nucleic acids. Two proteins be described as “homologous” or “non-homologous,” but the degree of amino acid conservation is quantitated by percent identity and percent similarity. Nucleic acid conservation is measured by the strict conservation of the bases adenine, thymine, guanine and cytosine in the primary nucleotide sequence. When describing nucleic acid conservation, conservation of the nucleic acid primary sequence is sometimes expressed as percent homology. In the same nucleic acid, one region may show a high percentage of nucleotide sequence conservation, while a different region shows no or poor conservation. It is not possible to infer nucleotide sequence conservation from an amino acid similarity score. Indeed, it is possible for two proteins to show domains that in one region are homologous, while other regions of the same protein the domains are non-homologous.

The term “isolated” when used in relation to a nucleic acid, as in “an isolated nucleic acid,” “an isolated oligonucleotide,” “isolated polynucleotide” or “isolated nucleotide sequence,” refers to a nucleic acid that is identified and separated from at least one contaminant nucleic acid with which it is ordinarily associated in its natural source. Isolated nucleic acid is present in a form or setting that is different from the form or setting of that nucleic acid found in nature. In contrast, non-isolated nucleic acids are found in the state in which they exist in nature. For example, a given DNA sequence (e.g., a gene) is found on the host cell chromosome in proximity to neighboring genes; RNA sequences, such as a specific mRNA sequence encoding a specific protein, are found in the cell in a mixture with numerous other mRNAs that encode a multitude of proteins. However, isolated nucleic acid encoding a given polypeptide includes, by way of example, such nucleic acid in cells ordinarily expressing the given protein where the nucleic acid is in a chromosomal location different from that of natural cells, or is otherwise flanked by a different nucleic acid sequence than that found in nature. This isolated nucleic acid, oligonucleotide, or polynucleotide is either single-stranded or double-stranded. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide is single-stranded). In other embodiments, the oligonucleotide or polynucleotide contains both the sense and anti-sense strands (i.e., the oligonucleotide or polynucleotide is double-stranded).

As used herein, the term “purified” or “to purify” refers to the removal of at least one contaminant from a sample. As used herein, the term “substantially purified” refers to molecules, either nucleic acids or amino acid sequences, that are removed from their natural environment, “isolated” or “separated,” and are largely free from other components with which they are naturally associated. An “isolated nucleic acid” or “isolated polypeptide” are therefore a substantially purified nucleic acid or substantially purified polypeptide. For example, antibodies are purified by removal of contaminating non-immunoglobulin proteins; they are also purified by the removal of non-specific immunoglobulin that does not bind to the target molecule. The removal of non-immunoglobulin proteins and/or the removal of immunoglobulins that do not bind to the target molecule results in an increase in the percent of target-reactive immunoglobulins in the sample. The removal of non-immunoglobulin proteins and/or the removal of immunoglobulins that do not bind to the target molecule results in an increase in the percent of target-reactive immunoglobulins in the sample (i.e., “enrichment” of an antibody). In another example, recombinant polypeptides are expressed in bacterial host cells and the polypeptides are purified by the removal of host cell proteins; the percent of recombinant polypeptides relative to all polypeptides in the sample is thereby increased.

Nucleic acid molecules (e.g., DNA or RNA) are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also can be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, in some embodiments, enhancer elements exert their effect even when located 3′ of the promoter element or the coding region. Transcription termination and polyadenylation signals are located 3′ or downstream of the coding region.

The term “gene” refers to a nucleic acid (e.g., DNA) sequence comprised of parts, that when appropriately combined in either a native or recombinant manner, provide some product or function. In some embodiments, genes comprise coding sequences necessary for the production of a polypeptide, while in other embodiments, the genes do not comprise coding sequences necessary for the production of a polypeptide. Examples of genes that do not encode polypeptide sequences include ribosomal RNA genes (rRNA) and transfer RNA (tRNA) genes. In preferred embodiments, genes encode a polypeptide or any portion of a polypeptide within the gene's “coding region” or “open reading frame.” In some embodiments, the polypeptide produced by the open reading frame of a gene displays functional activity or properties of the full-length polypeptide (e.g., enzymatic activity, ligand binding, signal transduction, etc.), while in other embodiments, it does not.

In addition to the coding region of the nucleic acid, the term “gene” also encompasses the transcribed nucleotide sequences of the full-length mRNA adjacent to the 5′ and 3′ ends of the coding region. These noncoding regions are variable in size, and typically extend for distances up to or exceeding 1 kb on both the 5′ and 3′ ends of the coding region. The sequences that are located 5′ and 3′ of the coding region and are contained on the mRNA are referred to as 5′ and 3′ untranslated sequences (5′ UT and 3′ UT). Both the 5′ and 3′ UT may serve regulatory roles, including translation initiation, post-transcriptional cleavage and polyadenylation. The term “gene” encompasses mRNA, cDNA and genomic forms of a gene.

In some embodiments, the genomic form or genomic clone of a gene contains the sequences of the transcribed mRNA, as well as other non-coding sequences which lie outside of the mRNA. The regulatory regions which lie outside the mRNA transcription unit are sometimes called “5′ or 3′ flanking sequences.” A functional genomic form of a gene must contain regulatory elements necessary for the regulation of transcription. The term “promoter/enhancer region” is usually used to describe this DNA region, typically but not necessarily 5′ of the site of transcription initiation, sufficient to confer appropriate transcriptional regulation. Used alone, the term “promoter” is sometimes used synonymously with “promoter/enhancer.” In some embodiments, the promoter is constitutively active, or while in alternative embodiments, the promoter is conditionally active (i.e., where transcription is initiated only under certain physiological conditions or in the presence of certain drugs). In some embodiments, the 3′ flanking region contains additional sequences which regulate transcription, especially the termination of transcription. “Introns” or “intervening regions” or “intervening sequences” are segments of a gene which are contained in the primary transcript (i.e., hetero-nuclear RNA, or hnRNA), but are spliced out to yield the processed mRNA form. In some embodiments, introns contain transcriptional regulatory elements such as enhancers. The mRNA produced from the genomic copy of a gene is translated in the presence of ribosomes to yield the primary amino acid sequence of the polypeptide.

As used herein, the term “regulatory element” refers to a genetic element which controls some aspect of the expression of nucleic acid sequences. For example, a promoter is a regulatory element that enables the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells, as well as viruses. Analogous control elements (i.e., promoters and enhancers) are also found in prokaryotes. The selection of a particular promoter and enhancer to be operably linked in a recombinant gene depends on what cell type is to be used to express the protein of interest. Some eukaryotic promoters and enhancers have a broad host range while others are functional only in a limited subset of cell types (for review see, Voss et al., Trends Biochem. Sci., 11:287 [1986] and Maniatis et al., Science 236:1237 [1987]). For example, the SV40 early gene enhancer is very active in a wide variety of mammalian cell types (Dijkema et al., EMBO J, 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; Mizushima and Nagata, Nuc. Acids. Res., 18:5322 [1990]), the long terminal repeats of the Rous sarcoma virus (Gorman et al., Proc. Natl. Acad. Sci. USA 79:6777 [1982]), and human cytomegalovirus (Boshart et al., Cell 41:521 [1985]). Some promoter elements serve to direct gene expression in a tissue-specific manner.

As used herein, the term “promoter/enhancer” denotes a segment of DNA which contains sequences capable of providing both promoter and enhancer functions (i.e., the functions provided by a promoter element and an enhancer element). For example, the long terminal repeats of retroviruses contain both promoter and enhancer functions. In some embodiments, the promoter/enhancer is “endogenous,” while in other embodiments, the promoter/enhancer is “exogenous,” or “heterologous.” An “endogenous” promoter/enhancer is one which is naturally linked with a given gene in the genome. An “exogenous” or “heterologous” promoter/enhancer is one placed in juxtaposition to a gene by means of genetic manipulation (i.e., molecular biological techniques such as cloning and recombination) such that transcription of the gene is controlled by the linked promoter/enhancer.

The presence of “splicing signals” on an expression vector often results in higher levels of expression of the recombinant transcript. Splicing signals mediate the removal of introns from the primary RNA transcript and consist of a splice donor and acceptor site (See e.g., Sambrook et al., Molecular Cloning: A Laboratory Manual, Second Edition, Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires the presence of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a nucleic acid sequence that directs both the termination and polyadenylation of the nascent RNA transcript. Efficient polyadenylation of the recombinant transcript is desirable as transcripts lacking a poly A tail are unstable and are rapidly degraded. In some embodiments, the poly A signal utilized in an expression vector is “heterologous,” while in other embodiments, it is “endogenous.” An endogenous poly A signal is one that is found naturally at the 3′ end of the coding region of a given gene in the genome. A heterologous poly A signal is one that is isolated from one gene and placed 3′ of another gene. A commonly used heterologous poly A signal is the SV40 poly A signal. The SV40 poly A signal is contained on a 237 bp BamHI/BclI restriction fragment and directs both termination and polyadenylation (See e.g., Sambrook et al., Molecular Cloning: A Laboratory Manual, Second Edition, Cold Spring Harbor Laboratory Press, New York [1989], pp.16.6-16.7).

The terms “in operable combination,” “in operable order,” “operably linked” and similar phrases when used in reference to nucleic acid herein are used to refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein, the terms “an oligonucleotide having a nucleotide sequence encoding a gene,” “polynucleotide having a nucleotide sequence encoding a gene,” and similar phrases are meant to indicate a nucleic acid sequence comprising the coding region of a gene (i.e., the nucleic acid sequence which encodes a gene product). In some embodiments, the coding region is present in a cDNA, while in other embodiments, the coding region is present in genomic DNA or RNA form. When present in a DNA form, the oligonucleotide, polynucleotide or nucleic acid is either single-stranded (i.e., the sense strand or the antisense strand) or double-stranded. In some embodiments, suitable control elements such as enhancers/promoters, splice junctions, polyadenylation signals, etc. are placed in close proximity to the coding region of the gene if needed to permit proper initiation of transcription and/or correct processing of the primary RNA transcript. Alternatively, the coding region utilized in the expression vectors of the present invention contains endogenous enhancers/promoters, splice junctions, intervening sequences, polyadenylation signals, etc. or a combination of both endogenous and exogenous control elements.

As used herein, the terms “nucleic acid molecule encoding,” “DNA sequence encoding,” and “DNA encoding” and similar phrases refer to the order or sequence of deoxyribonucleotides along a strand of deoxyribonucleic acid encoding a particular polypeptide. The order of the deoxyribonucleotides determines the order of the amino acids in the polypeptide chain. The DNA sequence thus codes for the amino acid sequence.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of the mRNA. Gene expression regulation often occurs at many stages. “Up-regulation” or “activation” refers to regulation that increases the production of gene expression products (i.e., RNA or protein), while “down-regulation” or “repression” refers to regulation that decreases mRNA or protein production. Molecules (e.g., transcription factors) that are involved in up-regulation or down-regulation are often called “activators” and “repressors,” respectively.

As used herein, the term “vector” is used in reference to nucleic acid molecules that transfer DNA segment(s) from one cell to another. The term “vehicle” is sometimes used interchangeably with “vector.” In some embodiments, a vector “backbone” comprises those parts of the vector which mediate its maintenance and enable its intended use (e.g., the vector backbone contains sequences necessary for replication, genes imparting drug or antibiotic resistance, a multiple cloning site, and possibly operably linked promoter/enhancer elements which enable the expression of a cloned nucleic acid). The cloned nucleic acid (e.g., such as a cDNA coding sequence, or an amplified PCR product) is inserted into the vector backbone using common molecular biology techniques. Vectors are often derived from plasmids, bacteriophages, or plant or animal viruses. A “cloning vector” or “shuttle vector” or “subcloning vector” contain operably linked parts which facilitate subcloning steps (e.g., a multiple cloning site containing multiple restriction endonuclease sites). A “recombinant vector” indicates that the nucleotide sequence or arrangement of its parts is not a native configuration, and has been manipulated by molecular biological techniques. The term implies that the vector is comprised of segments of DNA that have been artificially joined.

The term “expression vector” as used herein refers to a recombinant DNA molecule containing a desired coding sequence and operably linked nucleic acid sequences necessary for the expression of the operably linked coding sequence in a particular host organism (e.g., a bacterial expression vector, a yeast expression vector or a mammalian expression vector). Nucleic acid sequences necessary for expression in prokaryotes typically include a promoter, an operator (optional), and a ribosome binding site, often along with other sequences. Eukaryotic cells utilize promoters, enhancers, and termination and polyadenylation signals and other sequences which are different from those used by prokaryotes.

In some embodiments, eukaryotic expression vectors also contain “viral replicons” or “viral origins of replication.” Viral replicons are viral DNA sequences that allow for the extrachromosomal replication of a vector in a host cell expressing the appropriate replication factors. Some vectors replicate their nucleic acid to high copy numbers (e.g., vectors that contain either the SV40 or polyoma virus origin of replication replicate to high “copy number” (up to 10⁴ copies/cell) in cells that express the appropriate viral T antigen). Other vectors replicate their nucleic acid in low copy numbers (e.g., vectors that contain the replicons from bovine papillomavirus or Epstein-Barr virus replicate extrachromosomally at “low copy number” (˜100 copies/cell). The viral origins of replication listed above are not limiting, as the art is aware of other origins of replication that are commonly used in eukaryotic expression vectors.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and in some embodiments, include gene sequences found in that animal so long as the introduced gene does not reside in the same location as does the naturally-occurring gene.

The terms “overexpression” and “overexpressing” and grammatical equivalents are used in reference to levels of mRNA or protein where the level of expression of the mRNA or protein is higher than that typically observed in a given tissue in a control or non-transgenic animal. Levels of mRNA or protein are measured using any of a number of techniques known to those skilled in the art. For example, in some embodiments mRNA levels are assayed using methods such as Northern blot analysis (however, it is not intended that the present invention be limited to Northern analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample is used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into cells. Transfection can be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Mammalian cell transfection techniques are common in the art, and are described in many sources (See, e.g., Ausubel et al. (eds.), Current Protocols in Molecular Biology, Vol. 1-4, John Wiley & Sons, Inc., New York [1994]).

The term “stable transfection” or “stably transfected” refers to the introduction and integration of foreign DNA into the genome of the transfected cell. The term “stable transfectant” refers to a cell which contains stably integrated foreign DNA within its own genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

The term “calcium phosphate co-precipitation” refers to a technique for the introduction of nucleic acids into a eukaryotic cell, and most typically mammalian cells. The uptake of nucleic acids by cells is enhanced when the nucleic acid is presented as a calcium phosphate-nucleic acid co-precipitate. Various modifications of the original technique of Graham and van der Eb (Graham and van der Eb, Virol., 52:456 [1973]) are known in which the conditions for the transfection of a particular cell type has been optimized. The art is well aware of these various methods.

The term “transformation” has various meanings, depending on its usage. In one sense, the term “transformation” is used to describe the process of introduction of foreign DNA into prokaryotic cells (i.e., bacterial cells), and most frequently E. coli strains. Bacterial cell transformation can be accomplished by a variety of means well known in the art, including the preparation of “competent” bacteria by the use of calcium chloride, magnesium chloride or rubidium chloride, and electroporation. When a plasmid is used as the transformation vector, the plasmid typically contains a gene conferring drug resistance, such as the genes encoding ampicillin, tetracycline or kanamycin resistance. Bacterial transformation techniques are common in the art, and are described in many sources (e.g., Cohen et al., Proc. Natl. Acad. Sci. USA 69: 2110-2114 [1972]; Hanahan, J. Mol. Biol., 166:557-580 [1983]; Sambrook et al. (eds.), Molecular Cloning: A Laboratory Manual, Second Edition, Volumes 1-3, Cold Spring Harbor Laboratory Press, NY, [1989]; Ausubel et al. (eds.), Current Protocols in Molecular Biology, Vol. 1-4, John Wiley & Sons, Inc., New York [1994]).

“Transformation” also describes the physiological process by which a normal eukaryotic cell acquires the phenotypic qualities of a malignant cell. Such properties include, but are not limited to the ability to grow in soft agar, the ability to grow in nutrient poor conditions, rapid proliferation, and the loss of contact inhibition. A eukaryotic cell which is “transformed” displays the properties of malignant cells. In some embodiments, eukaryotic cells acquire their transformed phenotype in vivo, while in other embodiments, the cells are artificially transformed in culture.

As used herein, the term “selectable marker” refers to the use of a gene that encodes an enzymatic activity that confers the ability to grow in medium lacking what would otherwise be an essential nutrient (e.g., the HIS3 gene in yeast cells); in addition, in some embodiments, a selectable marker confers resistance to an antibiotic or drug upon the cell in which the selectable marker is expressed. Furthermore, some selectable markers are “dominant.” Dominant selectable markers encode an enzymatic activity that is detectable in any suitable eukaryotic cell line. Examples of dominant selectable markers include the bacterial aminoglycoside 3′ phosphotransferase gene (i.e., the neo gene) that confers resistance to the drug G-418 in mammalian cells, as well as the bacterial hygromycin G phosphotransferase (hyg) gene that confers resistance to the antibiotic hygromycin, and the bacterial xanthine-guanine phosphoribosyl transferase gene (i.e., the gpt gene) that confers the ability to grow in the presence of mycophenolic acid. The use of non-dominant selectable markers must be in conjunction with a cell line that lacks the relevant enzyme activity. Examples of non-dominant selectable markers include the thymidine kinase (tk) gene (used in conjunction with tk⁻ cell lines), the CAD gene (used in conjunction with CAD-deficient cells) and the mammalian hypoxanthine-guanine phosphoribosyl transferase (hprt) gene (used in conjunction with hprt⁻ cell lines). A review of the use of selectable markers in mammalian cell lines is provided in Sambrook et al., Molecular Cloning: A Laboratory Manual, Second Edition, Cold Spring Harbor Laboratory Press, New York (1989), at pp.16.9-16.15.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the terms “host,” “expression host,” and “transformant” refer to organisms and/or cells which harbor an exogenous DNA sequence (e.g. via transfection), an expression vector or vehicle, as well as organisms and/or cells that are suitable for use in expressing a recombinant gene or protein. It is not intended that the present invention be limited to any particular type of cell or organism. Indeed, it is contemplated that any suitable organism and/or cell will find use in the present invention as a host.

As used herein, the term “eukaryote” refers to organisms distinguishable from “prokaryotes.” It is intended that the term encompass all organisms with cells that exhibit the usual characteristics of eukaryotes such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “antibody” (or “antibodies”) refers to any immunoglobulin that binds specifically to an antigenic determinant, and specifically, binds to proteins identical or structurally related to the antigenic determinant which stimulated their production. Thus, antibodies are useful in methods to detect the antigen which stimulated their production. Monoclonal antibodies are derived from a single clone of B lymphocytes (i.e., B cells), and are generally homogeneous in structure and antigen specificity. Polyclonal antibodies originate from many different clones of antibody-producing cells, and thus are heterogenous in their structure and epitope specificity, but all recognize the same antigen. In some embodiments, purified monoclonal and/or polyclonal antibodies are used, while in other embodiments, crude preparations are used. For example, in some embodiments, polyclonal antibodies in crude antiserum are utilized. It is intended that the term “antibody” encompass any immunoglobulin (e.g., IgG, IgM, IgA, IgE, IgD, etc.) obtained from any source (e.g., humans, rodents, lagomorphs, non-human primates, caprines, bovines, equines, ovines, etc.).

As used herein, the terms “auto-antibody” or “auto-antibodies” refer to any immunoglobulin that binds specifically to an antigen that is native to the host organism that produced the antibody (i.e., the antigen is directed against “self” antigens). The presence of auto-antibodies is referred to herein as “autoimmunity.”

As used herein, the term “antigen” is used in reference to any substance that is capable of being recognized by an antibody. It is intended that this term encompass any antigen and “immunogen” (i.e., a substance which induces the formation of antibodies). Thus, in an immunogenic reaction, antibodies are produced in response to the presence of an antigen or portion of an antigen. The terms “antigen” and “immunogen” are used to refer to an individual macromolecule or to a homogeneous or heterogeneous population of antigenic macromolecules. It is intended that the terms antigen and immunogen encompass protein molecules or portions of protein molecules, which contains one or more epitopes. In many cases, antigens are also immunogens, thus the term “antigen” is often used interchangeably with the term “immunogen.” An immunogenic substance can be used as an antigen in an assay to detect the presence of appropriate antibodies in the serum of the immunized animal.

As used herein, the terms “antigen fragment” and “portion of an antigen” and the like are used in reference to a portion of an antigen. Antigen fragments or portions occur in various sizes, ranging from a small percentage of the entire antigen to a large percentage, but not 100%, of the antigen. However, in situations where at least a portion of an antigen is specified, it is contemplated that the entire antigen is also present (although it is not required that the entire antigen be present). In some embodiments, antigen fragments and/or portions do not comprise an “epitope” recognized by an antibody, while in preferred embodiments, antigen fragments and/or portions do comprise an epitope that is recognized by an antibody (e.g., an antibody of interest). In some embodiments, antigen fragments and/or portions are not immunogenic, while in preferred embodiments, antigen fragments and/or portions are immunogenic.

The terms “antigenic determinant” and “epitope” as used herein refer to that portion of an antigen that makes contact with a particular antibody variable region. When a protein or fragment (or portion) of a protein is used to immunize a host animal, numerous regions of the protein may induce the production of antibodies which bind specifically to a given region or three-dimensional structure on the protein (i.e., these regions or structures are referred to as antigenic determinants). In some embodiments, an antigenic determinant (e.g., a fragment of an antigen) competes with the intact antigen (i.e., the “immunogen” used to elicit the immune response) for binding to an antibody.

The terms “specific binding” and “specifically binding” when used in reference to the interaction between an antibody and an antigen describe an interaction that is dependent upon the presence of a particular structure (i.e., the antigenic determinant or epitope) on the antigen. In other words, the antibody recognizes and binds to a protein structure unique to the antigen, rather than binding to all proteins in general (i.e., non-specific binding).

As used herein the term “immunogenically-effective amount” refers to that amount of an immunogen required to invoke the production of protective levels of antibodies in a host upon vaccination.

As used herein, the term “adjuvant” is defined as a substance which enhances the immunogenicity of a co-administered antigen. If adjuvant is used, it is not intended that the present invention be limited to any particular type of adjuvant—or that the same adjuvant, once used, be used for all subsequent immunizations. The present invention contemplates many adjuvants, including but not limited to, keyhole limpet hemocyanin (KLH), agar beads, aluminum hydroxide or phosphate (alum), Freund's adjuvant (incomplete or complete), Quil A adjuvant and Gerbu adjuvant (Accurate Chemical and Scientific Corporation), and bacterins (i.e., killed preparations of bacterial cells, especially mycoplasma).

As used herein, the term “immunoassay” refers to any assay that uses at least one specific antibody for the detection or quantitation of an antigen. Immunoassays include, but are not limited to, Western blots, enzyme-linked immunosorbent assays (ELISAs or EIAs), radioimmunoassays (RIAs), and immunofluorescence assays (IFAs). Furthermore, many different ELISA formats are known to those in the art, and which find use in the present invention. However, it is not intended that the present invention be limited to these assays. Thus, other antigen-antibody reactions find use in the present invention, including but not limited to “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

As used herein, the term “ELISA” refers to enzyme-linked immunosorbent assay (or EIA). Numerous ELISA methods and applications are known in the art, and are described in many references (See e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]).

One ELISA method finding use with the present invention is a “direct ELISA.” In this embodiment, an antigen is immobilized to a solid support (e.g., a microtiter plate well), and is detected directly using an enzyme-conjugated antibody specific for the antigen. In an alternative embodiment, an “indirect ELISA” is used. In this embodiment, an antigen is immobilized to a solid support (e.g., a microtiter plate well) as in the direct ELISA, but is detected indirectly by first adding an antigen-specific antibody, followed by the addition of a detection antibody specific for the antibody that specifically binds the antigen, also known as “species-specific” antibodies (e.g. a goat anti-rabbit antibody), which are commercially available (e.g., Santa Cruz Biotechnology; Zymed; and Pharmingen/Transduction Laboratories).

“Sandwich ELISAs” also find use with the present invention. In a sandwich ELISA, the antigen is immobilized on a solid support (e.g., a microtiter plate) via an antibody (i.e., a capture antibody) that is immobilized on the solid support and is able to bind the antigen of interest. Following the affixing of a suitable capture antibody to the immobilized phase, a sample is added to the microtiter plate well, followed by washing. If the antigen of interest is present in the sample, it is bound to the capture antibody present on the support. In some embodiments, the sandwich ELISA is a “direct sandwich” ELISA, in which the captured antigen is detected directly by using an enzyme-conjugated antibody directed against the antigen, while in alternative embodiments, the sandwich ELISA is an “indirect sandwich” ELISA, in which the captured antigen is detected indirectly by using an antibody directed against the antigen, which is then detected by another enzyme-conjugated antibody which binds the antigen-specific antibody, thus forming an antibody-antigen-antibody-antibody complex. Suitable reporter reagents are then added to detect the third antibody. Alternatively, in other embodiments, any number of additional antibodies are added as necessary to detect the antigen-antibody complex. In some embodiments, these additional antibodies are also labelled or tagged to permit their visualization and/or quantitation.

As used herein, the term “capture antibody” refers to an antibody that is used in a sandwich ELISA (or other “sandwich” type immunoassays) to bind (i.e., capture) an antigen in a sample prior to detection of the antigen. Biotinylated capture antibodies are typically used in the present invention in conjunction with avidin-coated solid support. Another antibody (i.e., the detection antibody) is then used to bind and detect the antigen-antibody complex, in effect forming a “sandwich” comprised of antibody-antigen-antibody (i.e., a sandwich ELISA).

As used herein, a “detection antibody” is an antibody which carries on it a means for visualization or quantitation, which is typically a conjugated enzyme moiety that yields a colored or fluorescent reaction product following the addition of a suitable substrate. Conjugated enzymes commonly used with detection antibodies in ELISAs include horseradish peroxidase, urease, alkaline phosphatase, glucoamylase and β-galactosidase. In some embodiments, detection antibodies are directed against the antigen of interest, while in other embodiments, they are not. Typically, detection antibodies are anti-species antibodies. Alternatively, the detection antibody is prepared with a label such as biotin, a fluorescent marker, or a radioisotope, and is detected and/or quantitated using this label.

As used herein, the term “reporter reagent” or “reporter molecule” or “detection substrate” or “detection reagent” is used in reference to reagents which permit the detection and/or quantitation of an antibody bound to an antigen. For example, in preferred embodiments, a reporter reagent is a colorimetric substrate for an enzyme that has been conjugated to an antibody. A suitable substrate in the presence of the antibody-enzyme conjugate results in the production of a calorimetric or fluorimetric signal. Other reporter reagents include, but are not limited to, radioactive compounds. This definition also encompasses the use of biotin and avidin-based compounds (e.g., including but not limited to neutravidin and streptavidin) as part of the detection system.

As used herein, the term “signal” is used generally in reference to any detectable process that indicates that a reaction has occurred, for example, binding of antibody to antigen. It is contemplated that signals in the form of radioactivity, fluorimetric or calorimetric products/reagents find use with the present invention. In some embodiments, the signal is assessed quantitatively, while in other embodiments, the signal is assessed qualitatively (or both quantitatively and qualitatively).

As used herein, the term “amplifier” is used in reference to a system which enhances the signal in a detection method, such as an ELISA (e.g., an alkaline phosphatase amplifier system used in an ELISA).

As used herein, the term “solid support” is used in reference to any solid material to which reagents such as antibodies, antigens, and other components may be attached. For example, in the ELISA method, the wells of microtiter plates provide solid supports. Other examples of solid supports include microscope slides, coverslips, beads, particles, cell culture flasks, as well as many other suitable items.

As used herein, the term “kit” is used in reference to a combination of reagents and other materials which facilitates an assay and the analysis of a sample. In some embodiments, the immunoassay kits of the present invention include suitable capture antibody, reporter antibody, antigen, detection reagents and an amplifier system. Furthermore, in some embodiments, the kit also includes, but is not limited to, apparatus for sample collection, sample tubes, holders, trays, racks, dishes, plates, instructions to the kit user, solutions or other chemical reagents, and samples to be used for standardization, normalization, and/or control samples.

The terms “Western blot,” “Western immunoblot” “immunoblot” and “Western” refer to the immunological analysis of protein(s), polypeptides or peptides that have been immobilized onto a membrane support. The proteins are first resolved by polyacrylamide gel electrophoresis (i.e., SDS-PAGE) to separate the proteins, followed by transfer of the protein from the gel to a solid support, such as nitrocellulose, polyvinylidene difluoride (PVDF) or a nylon membrane. The immobilized proteins are then exposed to an antibody having reactivity towards an antigen of interest. The binding of the antibody (i.e., the primary antibody) is detected by use of a secondary antibody which specifically binds the primary antibody. The secondary antibody is typically conjugated to an enzyme which permits visualization of the antigen-antibody complex by the production of a colored reaction product or catalyzes a luminescent enzymatic reaction (e.g., the ECL reagent, Amersham).

The term “sample” as used herein is used in its broadest sense. The term “sample” as used herein refers to any type of material obtained from humans or other animals (e.g., any bodily fluid or tissue), cell or tissue cultures, cell lines, or a culture of microorganisms. “Sample” also encompasses food and feed (whether solid or liquid), media (whether solid or liquid) for the growth and maintenance of microorganisms and cell cultures, equipment and its components (e.g., dialysis, intravenous, and nasogastric tubing), disposable, as well as reusable patient care items (including catheters), environmental surfaces, soil, water and other fluids, and reagents (e.g., buffers). A biological sample suspected of containing nucleic acid encoding a protein of interest (e.g., Minn1) encompasses a cell or cells, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein typically comprises a cell, a portion of a tissue, and/or an extract containing one or more proteins and the like.

As used herein, the term “host cell” refers to any cell capable of harboring an exogenous nucleic acid or gene product. In some embodiments, the host cell also transcribes and/or translates and expresses a gene contained on the exogenous nucleic acid. It is intended that the exogenous nucleic acid be obtained from any suitable source. In some embodiments, it is produced synthetically, while in other embodiments, it is produced by another cell or organism. In addition, in some embodiments, the exogenous nucleic acid is subjected to replication, while in other embodiments, it is not. For example, the bacterium Escherichia coli strain BL21 is suitable for use as a host cell for a bacterial expression vector encoding the Minn1 polypeptide.

As used herein, a “drug” refers to any molecule of any composition, including protein, peptide, nucleic acid, organic molecule, inorganic molecule, or combinations of molecules, biological or non-biological, which are capable of producing a physiological response. As used herein, a “drug” provides at least one beneficial response in the cure, mitigation, treatment or prevention of a disease, condition or disorder (e.g., to eliminate a tumor cell). A compound is considered a “drug candidate” if it is not yet known if that compound will provide at least one beneficial response in the cure, mitigation, treatment or prevention of a disease, disorder or condition.

As used herein, the term “in vitro” refers to an artificial environment and to processes or reactions that occur within an artificial environment. The term “in vivo” refers to the natural environment (e.g., in an animal or in a cell) and to processes or reactions that occur within a natural environment. The definition of an in vitro versus in vivo system is particular for the system under study. For example, as used herein, studies of the ability of Ras and Minn1 to form a physical interaction using bacterially produced, purified proteins is an in vitro system. Conversely, the study of the ability of Ras and Minn1 proteins to form a physical interaction within a mammalian cell following the transient transfection of expression vectors is an in vivo experimental system.

As used herein, the term “subject” refers to any animal being examined, studied or treated. It is not intended that the present invention be limited to any particular type of subject. It is contemplated that multiple organisms will find use in the present invention as subjects. In some embodiments, humans are the preferred subject.

As used herein, the term “inhibit” refers to the act of diminishing, suppressing, alleviating, preventing, reducing or eliminating. For example, in some embodiments, a treatment that inhibits a tumor completely eradicates the tumor, reduces the tumor size, prevents further tumor growth, and/or reduces the rate of tumor growth. The term “inhibit” applies equally to both in vitro and in vivo systems.

As used herein, the term “DNA-dependent DNA polymerase” refers to a DNA polymerase that uses a single strand of deoxyribonucleic acid (DNA) as a template for the synthesis of a complementary and antiparallel DNA strand.

As used herein, the term “RNA-dependent DNA polymerase” refers to a DNA polymerase that uses ribonucleic acid (RNA) as a template for the synthesis of a complementary and antiparallel DNA strand. The process of generating a DNA copy of an RNA molecule is commonly termed “reverse transcription,” and the enzyme that accomplishes this is a “reverse transcriptase.” In some cases, a reverse transcriptase also contains ribonuclease activity. Furthermore, some DNA polymerase enzymes contain both DNA-dependent as well as RNA-dependent DNA polymerase activity. These dual-activity polymerases are frequently used in RT-PCR reactions.

As used herein, a “thermostable” enzyme is, in its most general sense, an enzyme that retains activity at elevated temperatures. In some embodiments, a thermostable DNA-polymerase, as used in PCR reactions, retains polymerase activity at temperatures at or in excess of 90° C. However, it is not intended that the present invention be limited to thermostable enzymes with a specific range of activity. Rather, it is intended that the term encompass enzymes that are active at temperatures that are higher that the optimum temperature of mesophilic enzymes.

As used herein, the term “tumor” refers to a neoplasia, and most frequently, to a malignant neoplasia.

As used herein, a “solid tumor” is a tumor that forms a mass with defined borders. As used herein, “tumor tissue” refers to tissue (including cells) from a solid tumor.

As used herein, the term “non-tumorigenic tissue” is tissue (including cells) that is free of tumor, or does not otherwise give rise to tumor tissue.

As used herein, the terms “local” or “localized” and the like refer to confinement to a small area, a single tissue (e.g., ovarian tissue), a single organ (e.g., a lung) or other structure (e.g., a solid tumor).

As used herein, the term “localized delivery” is delivery of an agent (e.g., a gene therapy agent or a drug) to a small area, a single tissue, a single organ or other specific structure (e.g., a solid tumor). For example, localized delivery of a gene therapy agent to a single site (e.g., a solid tumor) in a subject is typically achieved by injection into that site.

As used herein, the term “systemic” refers to multiple sites, tissues or organs in an organism, or to the entire organism. Use of the word “systemic” generally indicates involvement of the circulatory and/or lymphatic systems.

As used herein, the term “systemic delivery” (in contrast to localized delivery) is delivery of an agent (e.g., a drug) to multiple sites, tissues or organs in an organism, or to the entire organism via the circulatory system following an intravenous injection, or via gastrointestinal absorption of an orally administered agent.

As used herein, the term “surgical delivery” refers to the delivery of an agent (e.g., a gene therapy agent) by surgical means (i.e., by operation or some other invasive manipulation). Thus, in some embodiments, surgical techniques provide means for localized delivery of an agent.

As used herein, the terms “implant” or “implantation” or the like refer to the grafting or insertion of some device or structure into an organism. As used herein, a device (e.g., a capsule or chamber) for controlled or extended release of a therapeutic agent (e.g., a gene therapy agent) is implanted into a subject. The implantation of devices for the delivery of therapeutic agents offers the benefit of delivery to a localized area (i.e., not systemically), increased localized concentration of the agent, as well as extended and continuous release of the agent to the localized area

### DETAILED DESCRIPTION OF THE INVENTION

Following the activation of Ras protein, a biochemical signalling cascade is initiated which controls subsequent cellular responses. Generally speaking, any protein acting downstream of Ras in the Ras signaling cascade can be considered a “Ras effector.” However, as used herein, the term “Ras effector” is used more specifically to describe a protein which binds directly to Ras, and is itself activated by Ras following Ras activation. One of the most extensively studied Ras effectors is the serine/threonine kinase Raf, which is a component of the well-studied Ras/Raf/Mek/MAP-kinase cascade (Campbell et al., Oncogene 17:1395-1413 [1998]; and Malumbres and Pellicer, Front. Biosci., 3:d887-d912 [1998]).

Although Raf is one of the best studied Ras effectors, it is now realized that a diverse collection of other proteins are also able to bind the Ras protein, and activate the Ras/Raf/MEK/MAP-kinase signalling pathway as well as other signalling cascades. Currently, these Ras effectors include p120 GAP, Ra1GDS, phosphoinositol 3-kinase (PI3-kinase), AF-6/Rsb1/canoe, Rin-1, and the zeta isoform of protein kinase C (PKCζ) (Campbell et al., Oncogene 17:1395-1413 [1998]; and Vojtek and Der, J. Biol. Chem., 273:19925-19928 [1998]). Some Ras-effectors, for example, Raf-1 and PI3-kinase, are known to be oncoproteins in their own right and have well-characterized enzymatic activities (Moodie et al., Science 260:1658-1661 [1993]; Vojtek et al., Cell 74:205-214 [1993]; Zhang et al., Nature 364:308-313 [1993]; Rodriguez-Viciana et al., EMBO J., 15:2442-2451 [1996]; and Rodriguez-Viciana et al., Cell 89:457-467 [1997]). Other members of the Ras effector family are less well characterized (Malumbres and Pellicer, Front. Biosci., 3:d887-d912 [1998]; Ellis and Clark, Cellular Signalling, 12(7):425-434 [2000]; and Shields et al., Trends Cell Biol., 10:147-154 [2000]).

Despite the heterogeneity of Ras proteins and Ras effectors, these proteins share common elements which appear to be required for them to interact. Ras proteins share a core region of 8 amino acids in their N-termini, which is the site of effector binding, and is called the “effector domain.” Of the Ras effectors that bind to this small Ras domain, many share a common structural motif known as the “Ras association domain” (RA), which has been shown experimentally to be required in some effector proteins for association of the effector with the Ras-family protein (Ponting and Benjamin, Trends Biochem. Sci., 21:422-425 [1996]). However, the RA domain sequences are very divergent, and the RA domain is found in some, but not all, Ras effectors. Furthermore, the presence of an RA domain may not reliably predict the presence of Ras-binding proteins (Ponting and Benjamin, Trends Biochem. Sci., 21:422-425 [1996]).

Ras proteins have been best studied for their role in cell proliferation and tumorigenesis. However, a paradoxical observation regarding the function of Ras has recently emerged. Ras is not only a component of signaling pathways which control cell proliferation, but Ras also transduces signals which result in growth inhibition, growth arrest and/or apoptosis. Examples of this phenomenon are demonstrated in a variety of cellular systems, and include the ability of Ras to induce senescence (Serrano et al., Cell 88:593-602 [1997]), necrosis (Chi et al., Oncogene 18:2281-2290 [1999], apoptosis (Mayo et al., Science 278:1812-1815 [1997]; Chen and Faller, Oncogene 11:1487-1498 [1995]; and Joneson and Bar-Sagi, Mol. Cell. Biol., 19:5892-5901 [1999]), and terminal differentiation (Bar-Sagi and Feramisco, Cell 42:841-848 [1985]).

It is reasonable to postulate that a Ras-mediated growth inhibition signal is transmitted to the cell by one of two means. First, this Ras-mediated growth inhibition signal can use the same effector proteins that the mitogenic factors use to transmit cell proliferation signals, in such a way that the signal is recognized as an inhibitory signal and not a proliferation signal. For example, moderate oncogene activation has been shown to promote growth, but excessive, prolonged activation causes growth arrest and senescence (Sewing et al., Mol. Cell. Biol., 17:5588-5597 [1997]; and Zhu et al., Genes Dev., 12:2997-3007 [1998]). Alternatively, it is possible that the Ras-mediated inhibitory signal uses yet unidentified Ras effector(s), which function specifically to transmit only inhibitory signals to the cell. In this case, Ras-effectors which act specifically in inhibitory growth signalling would have properties of tumor suppressor genes, and may contribute to tumorigenesis if rendered ineffective by deletion or mutation. However, an understanding of the mechanism(s) is not necessary in order to use the present invention, nor is it intended that the present invention be limited to any particular mechanism(s).

The signalling mechanisms behind Ras mediated growth inhibition and apoptosis remain poorly understood. The observation that oncoproteins are capable of promoting cell death as well as transformation has led to the hypothesis that the signalling pathways that drive apoptosis and proliferation are tightly coupled in order to protect against oncogenic transformation (Hueber and Evan, Trends Genet., 14:364-367 [1998]; and Guo and Hay, Curr. Opin. Cell Biol., 11:745-752 [1999]). Understanding how Ras subverts this balance in a successful tumor is critical to understanding the role of Ras in human cancer. Thus, it is the goal of the present invention to identify genes which are Ras-effectors, function in growth inhibition, and/or have tumor suppressor properties. Genes which fit this criteria are excellent candidates for development as anti-cancer therapeutics in the treatment of cancers which display elevated Ras activity.

For convenience, the remainder of the Detailed Description of the Invention is divided into the following sections:

I. Identification, Cloning and Sequencing of the Ras Interacting Gene and Protein Minn1;

II. Ras Binds Minn1 in vitro;

III. Ras Binds Minn1 in vivo in a GTP-Dependent Manner;

IV. Analysis of Minn1 Expression;

V. Minn1-mediated Apoptosis is Ras-dependent;

VI. Antibodies Directed Against Minn1;

VII. Pharmaceutical Compositions Comprising the Minn1 Gene for the Treatment of Cancer; and

VIII. Methods and Compositions for the Analysis of the Minn1 Gene, Transcript and Protein

I. Identification, Cloning and Sequencing of the Ras Interacting Gene and Protein Minn1

To identify novel Ras-interacting proteins, and thus candidate Ras-effector proteins, an electronic screen was undertaken to identify proteins containing the Ras-Association (RA) domain (Ponting and Benjamin, Trends Biochem. Sci., 21:422-425 [1996]), as exemplified by the RA domain of the mouse Norel Ras-effector protein (SEQ ID NO:3) (Vavvas et al., Jour. Biol. Chem., 273(10):5439-5442 [1998]). A tBLASTn search of the National Center for Biotechnology Information (NCBI) expressed sequence tag (EST) database using the Nore1 RA domain as the search query identified a 613 base pair human EST (GenBank Accession Number AA205984) encoding this motif.

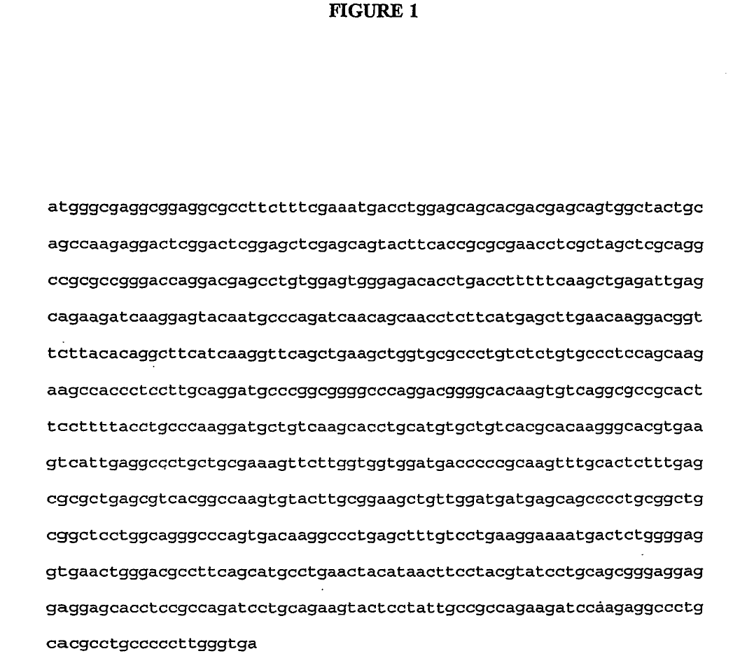

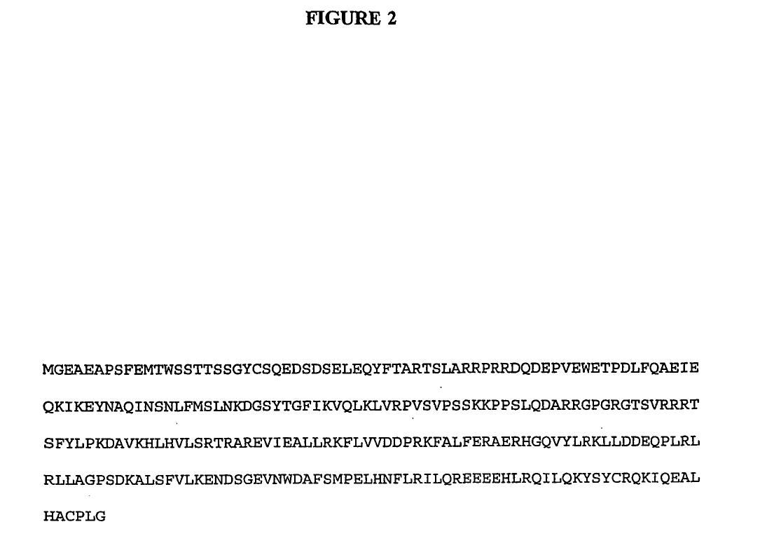

As this EST contained only a partial gene sequence, isolation and sequencing of the full length gene sequence was undertaken. A PCR cloning strategy was used to isolate the full length gene sequence, which was called Minn1. The Minn1 cDNA is predicted to contain an 813 bp open reading frame (shown in FIG. 1 and SEQ ID NO:1) encoding a 270 amino acid protein (shown in FIG. 2 and SEQ ID NO:2).

The 270 amino acid sequence predicted by the cDNA open reading frame was used to search NCBI GenBank. This search showed the 270 amino acid protein of the present invention to be novel. This search also identified protein sequences encoding a 270 amino acid protein which differ from the protein of the present invention at amino acid position 61. The protein of the present invention contains a phenylalanine at position 61, while the proteins described in these references contain a serine at position 61 (Dammann et al., Nature Genetics 25:315-319 [2000]; and GenBank Accession Numbers AF040703, AF132676, AF061836 and NM_{—}007182).

Analysis of genomic sequence databases using the Minn1 cDNA showed that the gene is localized to human chromosome 3p21.3 (GenBank Accession Number AC002481). Significantly, this genomic region is frequently deleted or rearranged in human lung and ovarian carcinomas (Fullwood et al., Cancer Res.,59:4662-4667 [1999]), and is contemplated to contain candidate tumor suppressor genes.

II. Ras Binds Minn1 In Vitro

The ability of Ras and Minn1 to interact in vitro was examined. These experiments used in vitro produced and purified maltose binding protein (MBP) fusion proteins containing the Minn1 Ras-Association (RA) domain or the Raf Ras-Binding-Domain (RBD), and purified Ras protein in a standard protein binding and co-precipitation assay. The MBP-Raf(RBD) protein was included in the binding assays to serve as a positive control for GTP-dependent Ras binding.

Briefly, bacterial expression vectors were generated as follows. The nucleotide sequence of the isolated Minn1 RA domain (spanning 211 amino acids, corresponding to amino acid positions 59-270) was generated as a PCR fragment and cloned in-frame into the pMa1-MBP fusion protein expression vector (NEB). An MBP-Raf(RBD) expression vector was constructed using a similar PCR strategy. Ras protein was produced by inducing an H-Ras bacterial expression construct in bacteria followed by differential denaturation and dialysis, as known in the art (See e.g., Campbell-Burk and Carpenter, Methods Enzymol., 255:3-13 [1995]). The recombinant MBP-Minn1(RA) and MBP-Raf(RBD) fusion proteins were produced in XL1-Blue Escherichia coli (Stratagene) and purified using maltose-conjugated Sepharose beads using standard techniques (See e.g., Clark et al., Jour. Biol. Chem., 272(34):20990-20993 [1997]). Purity and concentrations of the recombinant proteins were assessed by denaturing polyacrylamide gel electrophoresis (SDS-PAGE) followed by Coomassie Blue staining and comparison to known standards.

The in vitro binding assays contained purified MBP-Minn1(RA) or MBP-Raf(RBD) and purified recombinant H-Ras, which had been preloaded with either GTP or GDP, and were performed at 4° C. for 2 hours. After this time, the binding reactions were centrifuged and washed. Following the washing steps, the binding reactions were loaded and resolved using PAGE, blotted to a polyvinylidene difluoride (PVDF) membrane, and analyzed by Western immunoblotting using an anti-H-Ras monoclonal primary antibody (Quality Biotech, #146). Detection was accomplished using an alkaline phosphatase conjugated secondary antibody and ECL chemiluminescence reagent (Amersham). Nonspecific interaction between Ras and the MBP component of the fusion proteins was determined based upon the amount of Ras captured using an equivalent amount of purified MBP protein.

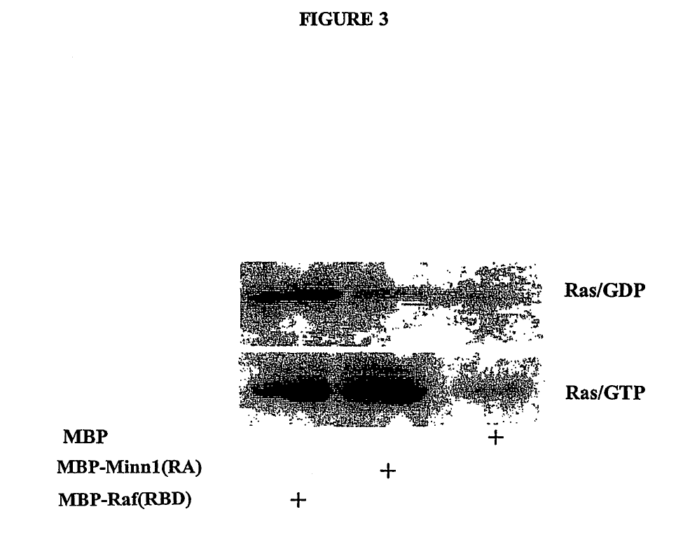

As shown in FIG. 3, Ras protein was co-precipitated with the MBP-Raf protein (i.e., the positive control), indicating a physical interaction between Ras and MBP-Raf. Furthermore, as expected, this interaction was GTP-dependent. Interestingly, the Minn1 protein behaved in a similar fashion (i.e., Ras protein was also co-precipitated with the MBP-Minn1 protein), indicating a physical interaction between Ras and MBP-Minn1, which was also GTP-dependent. Alone, the MBP peptide showed no affinity for the Ras protein either in the presence or absence of GTP.

As discussed above, Ras protein shuttles between an inactive, GDP-bound state and an active, GTP-bound state. Only the active, GTP bound form of Ras adopts the appropriate conformation to permit effector binding (Wittinghofer and Nassar, Trends Biochem. Sci., 21:488-491 [1996]). Therefore, if Minn1 is a Ras effector, its RA domain should bind GTP-bound Ras, but not GDP-bound Ras. As this experiment demonstrates that the Minn1 protein binds Ras in a GTP-dependent manner (i.e., a characteristic of Ras effector proteins), by this criteria, Minn1 is a Ras effector.

III. Ras Binds Minn1 In Vivo in a GTP-Dependent Manner

To confirm and complement the results observed in the in vitro binding assay, an in vivo binding assay was undertaken using recombinant FLAG-tagged Minn1 protein and hemagglutinin (HA)-tagged Ras proteins. These experiments used a standard co-transfection/co-precipitation protocol common in the art, using 293-T cells, a transformed human embryonal kidney cell line.

Expression vectors encoding two different forms of HA-tagged Ras protein were used in this assay, namely, a wild-type HA-H-Ras fusion protein and an HA-H-Ras containing a gain-of-function (G12V) mutation. This mutation is known to be oncogenic, and results in elevated Ras signalling activity (Clark and Der, in GTPases in Biology [eds. Dickey and Birmbauer], Springer-Verlag London Ltd., pp. 259-287 [1993]). This activated form of Ras typically shows greater than 70% association with GTP in vivo, while typically only 5% of wild-type Ras is bound to GTP. Thus, this mutant form of Ras is considered to be locked in an active conformation.

The in vivo binding assay was conducted by co-transfecting mammalian expression vectors encoding HA-H-Ras(WT) or HA-H-Ras(G12V) with an expression vector encoding FLAG-Minn1 into 293-T cells. After 48 hours, the cells were lysed, immunoprecipitated using anti-HA antibody-conjugated sepharose beads (BAbCO), washed and analyzed by Western immunoblotting using an anti-FLAG monoclonal antibody (M2 antibody, SIGMA) and an alkaline phosphatase conjugated secondary antibody with an ECL detection kit (Amersham).

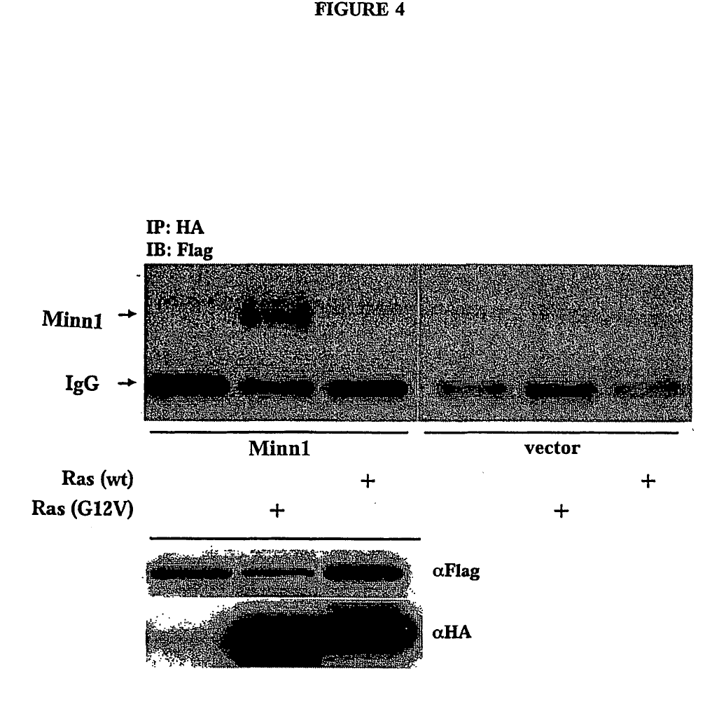

The results of this in vivo binding assay are shown in FIG. 4. As indicated in the top portion of Panel A, the FLAG-tagged Minn1 preferentially associated with the activated HA-Ras(G12V) protein, as compared to the HA-H-Ras(WT) protein. Analysis of the control FLAG-tag without any fused protein in the binding assay (top portion of Panel B) confirms that there is no non-specific affinity between the FLAG-tag and the Ras proteins. The Western blot in the lower portion of Panel A, in which anti-FLAG and anti-HA primary antibodies were used to confirm adequate expression of the fusion proteins in the 293-T cells. Thus, the result of this in vivo binding assay confirmed the observations made in the in vitro binding assay, where Minn1 preferentially bound to GTP-loaded Ras. The preferential association of Minn1 with the activated mutant H-Ras(G12V) further confirms that Minn1 is a candidate Ras-effector.

IV. Analysis of Minn1 Expression

The expression pattern of the Minn1 gene was investigated by Northern blotting using a variety of human tissues as well as in normal and ovarian cancer cell lines. The probe used in these experiments was a random-primed ³²P-dCTP labelled Minn1 cDNA.

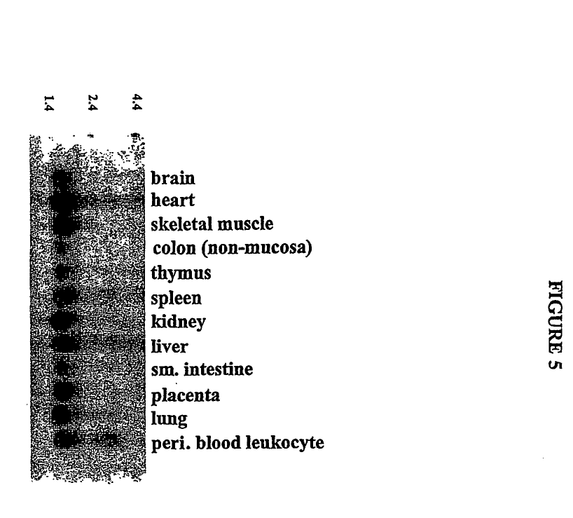

FIG. 5 shows a multiple human tissue Northern blot (Clontech) probed with the labelled Minn1 cDNA. As indicated in this Figure, a single predominant transcript corresponding to the Minn1 gene was present in the RNA of each tissue tested, and is present in varying degrees, with some tissues showing stronger Minn1 expression than other tissues.

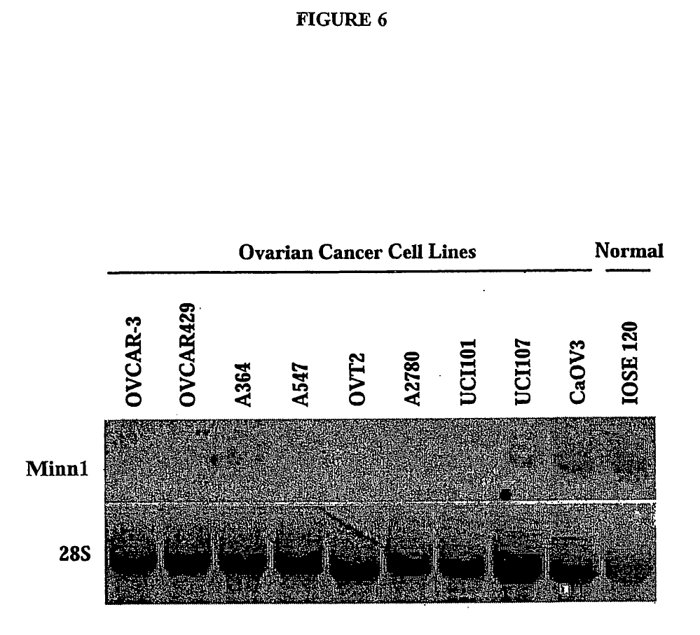

FIG. 6 shows a Northern blot of total RNA prepared from normal and ovarian tumor cell lines and probed using the same Minn1 cDNA probe. The cell lines included in this Northern were a non tumorigenic ovarian epithelial cell line IOSE-120, as well as ovarian tumor cell lines OVCAR-3, OVCAR429, A364, A547, OVT2, A2780, UC1101, UC1107 and CaOV3.

As indicated in FIG. 6, the non-transformed IOSE-120 cell line showed a single RNA species corresponding to the Minn1 transcript, while the majority of the ovarian cancer cell lines (6 of 9) did not show any Minn1 mRNA expression.

As the Minn1 gene maps to a region of the genome which is frequently deleted or rearranged in lung and ovarian tumors (Fullwood et al., Cancer Res., 59:4662-4667 [1999]), the Northern blot analysis of the ovarian cancer cell lines is of particular significance. Based on these results, it is contemplated that the Minn1 protein serves a function in all cells, but its loss causes or contributes to the oncogenic phenotype, as demonstrated by the loss of Minn1 expression in six out of nine transformed ovarian cell lines tested. This pattern indicates that the Minn1 gene has properties of a tumor suppressor gene.

Minn1 protein can be expressed as two different isoforms referred to as Minn1A and Minn1C, which is compatible with the exon structure of the gene.

V. Minn1-induced Apoptosis is Ras-dependent

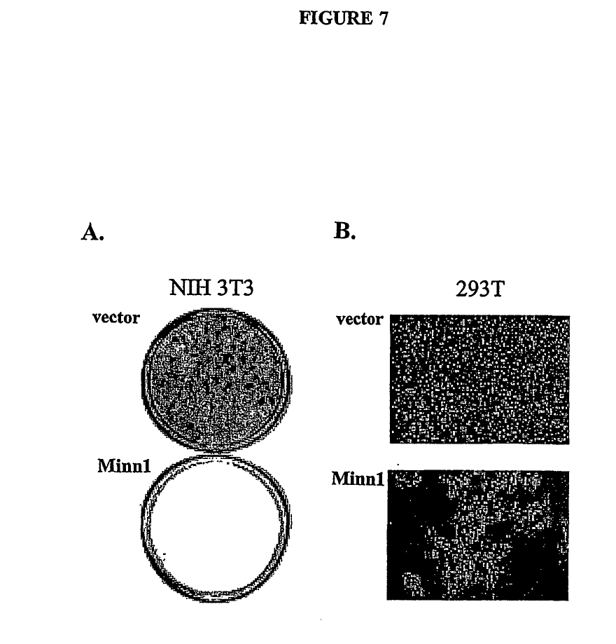

To examine the biological role of Minn1, construction of stable cell lines over-expressing Minn1 was attempted. To accomplish this, the Minn1 cDNA was cloned into an HA-tagged version of the pZIP-Neo SV(X)1 selectable mammalian expression vector (Cepko et al., Cell 37:1053-1062 [1984]), which was then transfected into N1H-3T3 cells at a concentration of 200 ng vector DNA per culture dish. The cells were then subjected to selection for 14 days in G418 at a concentration of 500 μg/ml. However, no cells in the Minn1 transfected dishes survived the selection (as shown in FIG. 7, Panel A, bottom portion). However, cells transfected with the empty pZIP-Neo control vector did form drug-resistant colonies following G418 selection (FIG. 7, Panel A, top portion), indicating that the transfection and reagents were effective. Moreover, co-transfection with activated Ras failed to rescue the cells (data not shown).

As the study of Minn1 activity in stably-transfected cell lines was not possible, the effects of Minn1 expression in transiently transfected cells was undertaken using 293-T cells, an embryonic human transformed kidney cell line (ATCC CRL No. 1573). The 293-T cells were transfected with 10 μg of the same Minn1 expression vector as above, and examined by phase contrast microscopy at 72 hours post-transfection, as shown in FIG. 7, Panel B. As indicated in FIG. 7, Panel B, the cells receiving the empty control vector (top portion) showed no growth inhibition, while the cells receiving the Minn1 expression vector (bottom portion) showed marked cell death.

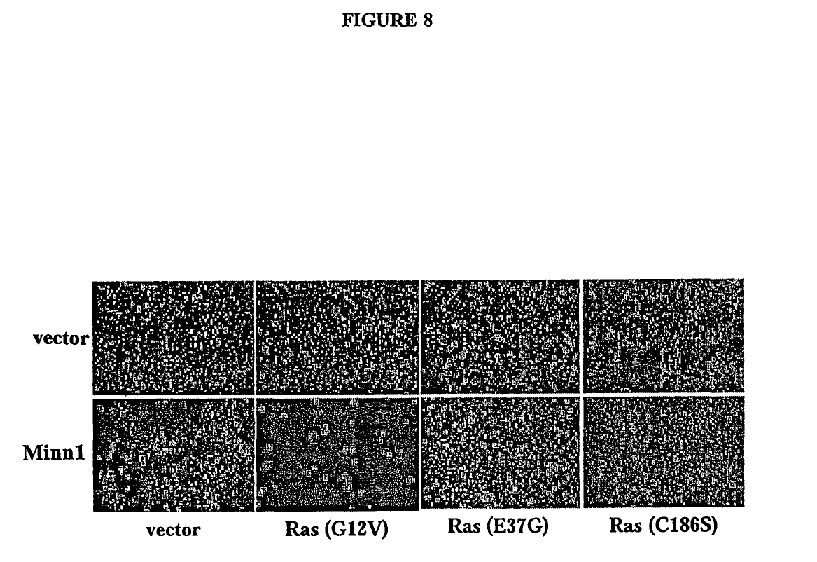

In order to determine whether Minn1 is a Ras-activated (i.e., Ras-dependent) tumor suppressor, Minn1-mediated growth inhibition was tested in the context of three different H-Ras mutants (White et al., Cell 80:533-541 [1995]; and Miyake et al., FEBS Lett., 378:15-18 [1996]). These mutants included an activated H-Ras (G12V), an effector domain mutant H-Ras (G12V/E37G), and a dominant negative H-Ras (Q61L/C186S). These mutants were used to determine whether activated Ras signalling stimulates tumor suppressor activity of Minn1. 293-T cells were transfected with 10 μg of pCDNA3 Minn1 expression vector and alternatively with 100 ng of each of the mutant H-Ras expression vectors. Parallel control transfections were done using the empty pCDNA control vector in combination with each of the H-Ras mutants. Cells were examined by phase contrast microscopy at 72 hours post-transfection, as shown in FIG. 8.

As indicated in this Figure, the presence of activated H-Ras (G12V) dramatically stimulated the growth inhibitory effects of Minn1. This stimulation was dependent upon an intact effector domain, as an effector domain mutant (H-Ras [G12V/E37G]) was unable to activate Minn1. The presence of a dominant-negative form of H-Ras (Q61L/C186S) also completely blocked the growth inhibitory properties of Minn1. Thus, the growth inhibition caused by Minn1 is Ras-dependent.

These results indicate that deregulated expression of Minn1 inhibits cell growth and survival, the growth inhibitory activity of Minn1 is dependent on Ras activity, and the Minn1 gene has activities consistent with tumor suppressor genes.

VI. Minn1 Mediates Cell Death by an Apoptotic Mechanism

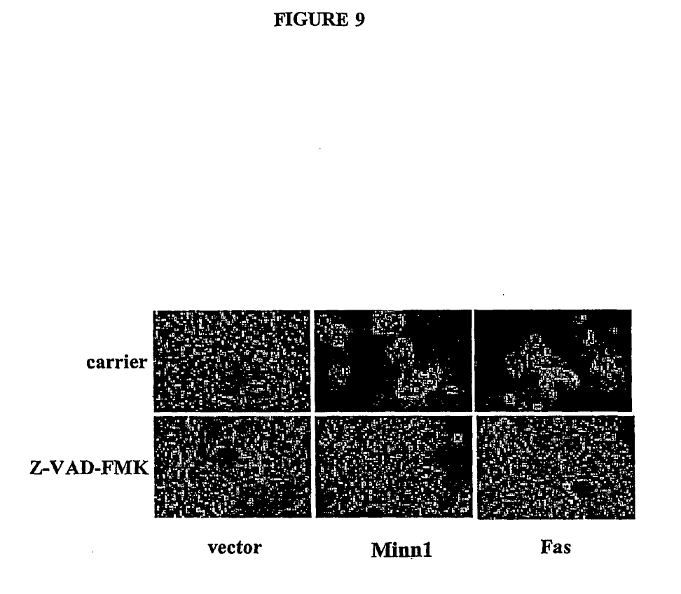

To examine the mechanism of the growth inhibition displayed by Minn1, the cell death observed in 293-T cells following transfection with a Minn1 expression vector was compared to the cell death observed following transfection with a Fas expression vector. Fas is a well-characterized inducer of apoptosis (Nagata, Annu. Rev. Genet., 33:29-55 [1999]). As shown in FIG. 9, cells transfected with either Minn1 or Fas each exhibited widespread cell death, as well as similar morphological changes, including membrane blebbing, a hallmark of apoptosis (Wyllie, Eur. J. Cell Biol., 73:189-197 [1997]).

Apoptosis requires the activation of caspase proteases (Stennicke and Salvesen, Biochim. Biophys. Acta 1477:299-306 [2000]). In order to confirm that the cell death observed in the Minn1 and Ras transfected cells was apoptotic cell death, the transfection experiments were repeated in the presence of the caspase inhibitor, z-VAD-fink (Calbiochem). The drug was added to the cells to a final concentration of 30 μM immediately after transfection and was maintained during subsequent medium changes. DMSO was used as the drug carrier, and was also included in transfections that contained no drug in order to normalize transfection conditions. As shown in FIG. 9, the ability of both Minn1 and Fas to induce cell death was severely reduced by the presence of z-VAD-fmk, indicating that Fas and Minn1 induce cell death by apoptosis.

VII. Antibodies Directed Against Minn1

The present invention provides polyclonal and monoclonal antibodies directed against the Minn1 protein. These antibodies find numerous uses, including diagnostic agents in the examination of tumor biopsy material, as well as in research on Minn1 structure, function and mechanism of action. These clinical/diagnostic and research methods include immunoassays, including but not limited to, Western immunoblotting, enzyme-linked immunosorbent assays (ELISAs), radioimmunoassays (RIAs), immunofluorescence assays (IFAs), immunoprecipitation, and immunohistochemistry and immunoaffinity purification, all of which are known in the art (See, e.g., Harlow and Lane (eds.), Antibodies: A Laboratory Manual, Cold Spring Harbor Laboratory Press [1988]; Ausubel et al. (eds.), Current Protocols in Molecular Biology, Vol. 1-4, John Wiley & Sons, Inc., New York [1994]; and Laurino et al., Ann. Clin. Lab Sci., 29(3):158-166 [1999]).

It is not intended that the present invention be limited to the antibody production methods provided below. Numerous methods for the production and purification of antibodies are well known in the art, and can be found in various sources (See e.g., Sambrook et al. (eds.), Molecular Cloning, Cold Spring Harbor Laboratory Press [1989]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). It is also not intended that the present invention be limited to any particular Minn1 antigen, nor any particular method for the production of Minn1 antigen. In some preferred embodiments, the antibodies of the present invention are directed against the internal peptide sequence RAREVIEALLRKFLVVDDPRK (SEQ ID NO:9). In some other preferred embodiments, the antibodies of the present invention are specifically directed against an isoform of Minn1. In some embodiments, the antibodies are directed against Minn1C, while in other embodiments, the antibodies are directed against Minn1A. In some preferred embodiments, the antibodies are directed against the sequence QEDSDSELEQYFTAR (SEQ ID NO:10), which corresponds to amino acid residues 24 to 36 in the Minn1 polypeptide sequence. However, it is not intended that the present invention be limited to antibodies that are directed against SEQ ID NOS:9, 10 or any other portions of Minn1, as other portions of Minn1 find use as immunogens. As those of skill in the art know, numerous protocols for the purification of polypeptides suitable for use as antigens are available.

Production of Minn1 antigen: A variety of protocols and reagents are useful in the production of substantially purified Minn1 polypeptide suitable for use as an antigen. In some embodiments of the present invention, the Minn1 antigen produced involves any portion of the Minn1 protein, where the portion is a minimum of 7 amino acids in length. In other embodiments, the Minn1 antigen is produced with or without a fusion protein tag (e.g., MBP or FLAG), while in still further embodiments, the Minn1 antigen is synthetic, recombinant or native. In additional embodiments, recombinant Minn1 antigen is produced in various cell types (e.g., bacterial cells or mammalian cells), while in still other embodiments, various expression vectors are used to drive expression of recombinant Minn1 protein within a cell. In further embodiments, the Minn1 antigen is purified by various methods (for example, including but not limited to, MBP or FLAG purification, as described herein). Indeed, it is not intended that the present invention be limited by the protocols provided in Examples 3 and 4 describing the production and purification of MBP- and FLAG-tagged Minn1 polypeptides. It is contemplated that any protocol which will produce a substantially purified Minn1 polypeptide will find use with the present invention. Such alternative protocols include the use of glutathione S-transferase (GST)-Minn1 fusion polypeptides, hemagglutinin (HA)-tagged Minn1 fusion polypeptides, polyhistidine (i.e., 6×His)-tagged Minn1 fusion polypeptides, thioredoxin-tagged Minn1 fusion polypeptides, and Minn1 polypeptides without any fused tag(s). In some embodiments, Minn1 polypeptides suitable for use as antigenic material are produced by synthetic chemical synthesis.

Various protocols for recombinant polypeptide production also find use in the present invention. In some embodiments of the present invention, various host systems are used to produce starting material for Minn1 purification. Such systems include insect cells with a baculovirus overexpression system (e.g., Sf9 or Sf21 cell lines), mammalian cell lines used in conjunction with vectors designed for recombinant polypeptide overexpression (expression vectors, e.g., pZipNeo and pCDNAFLAG), or mammalian cells or tissues for the purification of Minn1 polypeptide expressed from its endogenous (i.e., native) chromosomal location. The cultivation of the transformed, transfected or infected host of the invention is carried out in a medium under conditions most appropriate for the growth of that particular host cell. These media formulations and culture conditions are well known to those in the art.

Polyclonal Antisera Production: Briefly, in some embodiments of the present invention, Minn1 polypeptide, any portion thereof, either native, recombinant or synthetically produced, is used to raise polyclonal antisera in an animal (e.g. rabbit, rat, mouse, etc.). In some embodiments, standard technique is used to immunize a mammalian host, typically a rabbit, with the Minn1 antigen. In some embodiments, the antigen is conjugated to additional protein sequences (e.g., keyhole limpet hemocyanin [KLH]). In some embodiments, the antigen is mixed with an adjuvant (e.g. Freund's incomplete or complete adjuvant) prior to immunization. The dosage of the antigen administered per animal is typically between 0.1 and 10 mg when no adjuvant is used, and between 1.0 and 100 μg when an adjuvant is used, and is typically injected via intravenous, subcutaneous or intraperitoneal routes. The animals typically receive antigenic boosts at regular intervals (it is not intended that the interval of immunization be particularly limited). In preferred embodiments, immunization is carried out one to 10 times, preferably 2 to 5 times, at intervals of several days to several weeks, preferably at intervals of 2 to 5 weeks. Bleeds are obtained at regular intervals for analysis of antigen-specific immunoreactivity, using techniques common in the art (e.g., Western immunoblots).

Monoclonal Antibody Production: For preparation of monoclonal antibodies directed toward the Minn1 protein, or any portion thereof, any technique that provides for the production of antibody molecules by continuous cell lines in culture is used. These methods include but are not limited to the hybridoma technique originally developed by Köhler and Milstein (Köhler and Milstein, Nature 256:495-497 [1975]), as well as the trioma technique, the human B-cell hybridoma technique (See e.g., Kozbor et al. Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In some embodiments, the following protocol is used to produce a monoclonal antibody specific for a Minn1 protein of the present invention. It is not intended that the present invention be limited to the use of this or any other protocol, as numerous protocols for generating antibody-producing cells are known, and find use in the present invention.

Inoculation and Recovery of Antibody-Producing Cells: A mammalian animal host is immunized according to the protocol described above to produce polyclonal antisera. Subsequently, at 1 to 10 days, preferably 3 days, after the final immunization, antibody-producing cells are collected. Antibody-producing cells, including spleen cells, lymph node cells, peripheral blood cells, etc. are typically enumerated after isolation. In most embodiments, the spleen or local lymph node cells are used in the following steps.

Cell Fusion and Formation of Hybridoma Cell Lines: In order to obtain hybridomas which produce the monoclonal antibody, cell fusions between the antibody-producing cells described above and myeloma cells are performed. Preferably, cell strains used for this purpose are those with drug selectivity, cannot survive in HAT selective medium (i.e., containing hypoxanthine, aminopterin and thymidine) when infused, and are capable of surviving in this medium only when fused to antibody-producing cells. In some embodiments, mouse myeloma cell strains including but not limited to, P3X63Ag.8.U1(P3U1), Sp2/0, NS-1 are used as myeloma cells. Subsequently, the myeloma cells and the antibody-producing cells described above are subjected to cell fusion. In some embodiments, 1×10⁹ cells/ml of the antibody-producing cells and 1×10⁸ cells/ml of the myeloma cells are mixed together in equal volumes in cell culture medium (e.g., serum-free DMEM or RPMI-1640), and reacted in the presence of a cell fusion promoting agent. In some embodiments, polyethylene glycol with an average molecular weight of 1,500 Da is used as the cell fusion promoting agent. Alternatively, the antibody-producing cells and the myeloma cells are fused in a commercial cell fusion apparatus utilizing electric stimulation (e.g., electroporation).

Selection and Cloning of Hybridoma Lines: Following cell fusion, hybridomas are selected from the culture. In some embodiments, the cells are appropriately diluted in culture medium (e.g., RPMI-1640 medium containing with fetal bovine serum), and plated in microtiter plate wells at a density of about 2×10⁵ cells/well. A selective medium is added to each well, and the fused cells are incubated in this selective medium. As a result, about 14 days after the start of cultivation in the selective medium, hybridomas are produced.

Subsequently, screening is performed in order to determine the presence of the antibody of interest in the culture supernatant of the grown hybridomas. Any suitable method for screening of hybridomas finds use with the present invention. For example, in some embodiments, part of the culture supernatant of a well in which a hybridoma is grown is collected and subjected to enzyme immunoassay or radioimmunoassay.

Cloning of the fused cell is performed by the limiting dilution method or the like. Finally, the hybridoma of interest producing the monoclonal antibody of interest is established.

Production of Monoclonal Antibody: In some embodiments of the present invention, conventional cell culture methods or the abdominal dropsy formation method are employed for recovering the monoclonal antibody from the established hybridoma of interest (i.e., a monoclonal antibody-producing cell).

In the cell culture methods, the established hybridoma is cultured in a cell culture medium (e.g., RPMI-1640 or MEM medium, containing fetal bovine serum, or in a serum-free medium) under conventional culture conditions (e.g., at 37° C. in the presence of 5% CO₂) for 2 to 10 days. Then, the monoclonal antibody is then recovered from the culture supernatant.

In the abdominal dropsy formation method, about 1×10⁷ cells of the hybridoma are administered into the abdominal cavity of an animal syngeneic to the mammal from which the myeloma cells were derived, to thereby propagate the hybridoma greatly. One to two weeks thereafter, the abdominal dropsy or serum is collected.

Antibody Purification: Following the production of polyclonal or monoclonal antibodies, the antibodies are purified using any suitable method known in the art, including but not limited to Protein A/Protein G affinity, ammonium sulfate salting out, ion exchange chromatography, gel filtration, affinity chromatography, or any of these methods in combination, as known in the art (See, e.g., Sambrook et al. (eds.), Molecular Cloning, Cold Spring Harbor Laboratory Press [1989]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). In view of numerous alternative protocols known in the art for the production and purification of polyclonal and monoclonal antibodies, it is not intended that the present invention be limited to any particular method.

VIII. Pharmaceutical Compositions Comprising the Minn1 Gene for the Treatment of Cancer

In particularly preferred embodiments, the present invention provides a polypeptide that induces apoptosis and has tumor suppressor activity (i.e., the Minn1 polypeptide), and a gene encoding the polypeptide. It is contemplated that these compositions will find use as therapeutic agents for the treatment of cancer. It is contemplated that a recombinant Minn1 gene of the present invention has the ability to induce apoptosis in tumor cells, and more specifically, in tumor cells that contain elevated Ras activity. Indeed, it has been shown that a competition between transformation and cell death persists even in successful tumors, where high levels of apoptosis are still be detectable (Kerr and Currie, Br. J. Cancer 26:239-257 [1972]; and Lowe and Lin, Carcinogenesis 21:485-495 [2000]).

When compositions of the present invention are used as therapeutic agents in gene therapy for the treatment of cancer, it is not intended that the present invention be limited to any particular type of cancer. For example, it is contemplated that the present invention will be used to treat ovarian cancer. However, it is contemplated that the present invention will find use in the treatment of other cancers, including, but not limited to, lung cancer.

In one embodiment, the present invention is used to treat tumors that contain activated Ras mutations. In another preferred embodiment, the present invention is used to treat tumors that demonstrate loss or reduced expression of the endogenous Minn1 gene. In a most preferred embodiment, the present invention is used to treat tumors that contain activated Ras mutations and loss or reduced expression of the endogenous Minn1 gene.

In one embodiment, the present invention is used as a gene therapy agent to treat cancer. In one embodiment, the gene therapy agent of the present invention is delivered via a viral delivery system. In an alternative embodiment, the gene therapy agent of the present invention involves a non-viral delivery system.

Viral-mediated gene delivery has been shown to be an effective mechanism for gene delivery for use in gene therapy. Indeed, methods for viral-mediated gene therapy have recently been shown to be effective in human and non-human systems (Cavazzana-Calvo et al, Science 288:669-672 [2000]; Kay et al., Nature Genetics 24:257-261 [2000]; Amado and Chen, Science 285:674-676 [1999]; Burton et al., Proc. Natl. Acad. Sci. USA 96(22):12725-12730 [1999]; Zhang, Cancer Gene Ther., 6(2):113-138 [1999]; Connelly et al., Blood 91(9):3273-3281 [1998]; and Connelly et al., Blood 88(10):3846-3853 [1996]). A number of viruses have been demonstrated to be effective or potentially effective tools in recombinant gene delivery to subjects, including adenovirus (lentivirus) vectors, adeno-associated virus vectors, herpes virus vectors, vaccinia virus vectors, and retrovirus vectors. In some preferred embodiments, the recombinant viral vector comprising the Minn1 gene of the present invention comprises nucleic acid elements operably linked for the purpose of transcribing and translating the gene of the invention in tumor cells in a subject. In preferred embodiments, these nucleic acid elements consist of a nucleotide sequence encoding the Minn1 polypeptide, and operably linked promoter and enhancer elements for expression of the Minn1 gene. In some embodiments, these promoter/enhancer elements are widely active in all or many cell types, and direct constitutive expression of the gene (e.g., cytomegalovirus (CMV), SV40 or Rous sarcoma virus (RSV) promoter/enhancer sequences). In alternative embodiments, operably linked promoter/enhancer elements are restricted in activity to a single cell type or tissue (e.g., cardiac-specific, liver-specific or ovarian-specific promoter/enhancers) (Maniatis et al., Science 236:1237-1245 [1987]; Voss et al., Trends Biochem. Sci., 11:287 [1986]). In further embodiments, a promoter/enhancer element that imparts inducible (i.e., conditional) expression of an operably linked open reading frame (e.g., tetracycline inducible or repressible promoters) is used. Furthermore, in other embodiments, operably linked nucleotide sequences include sequences directing proper translation initiation, post-transcriptional splicing/editing, and/or polyadenylation. In still other embodiments, in addition to containing nucleotide sequences controlling the expression of the Minn1 gene, a viral gene therapy vector further contains the necessary nucleotide sequences for in vitro replication and propagation of the virus, production of infective virion particles, and sequences that impart stability of the DNA in a cellular host (although many viral functions require the presence of a “helper virus”). Collectively, such sequences are sometimes referred to as the viral “backbone.”

In alternative embodiments, non-viral delivery systems are used to deliver the Minn1 gene as a gene therapy agent. Non-viral delivery means include gene delivery by direct application of the nucleic acid to cells or tissues, or the use of phospholipid vesicles such as liposomes (Mahato et al., Adv. Genet., 41:95-156 [1999]).

The use of phospholipids (i.e., liposomes) is well documented to be an effective means of delivery of nucleic acid to a host cell. Thus, in some embodiments, nucleic acid of the present invention is enclosed in phospholipid vesicles such as liposomes, and the resultant liposomes administered to a subject, or to the tumor of the subject. Liposomes are biodegradable vesicles containing an internal aqueous region surrounded by a lipid bilayer. This structure is able to encapsulate materials (e.g., at least one gene of the present invention). By mixing at least one gene of the present invention with phospholipid starting material under appropriate conditions, a liposome-gene complex forms. Subsequently, when this complex is cultured with cells or administered to cells in a subject, the gene(s) in the complex is taken into the cells (i.e., via lipofection).

In still other embodiments, beads (e.g., DYNAFECT beads) coated with antibodies specific for defined cell surface antigens are used to deliver or enhance the transmembrane uptake of nucleic acid (Bildirici et al., Nature 405:298 [2000]). This process, also known as immunoporation, delivers DNA to cells at a high rate of efficiency, and offers the added benefit of targeting the particular cells to receive the gene of interest (Le., the Minn1 gene) in a mixed population of cells. In further embodiments, this technology is used to directly deliver Minn1 protein of the present invention to the site of a tumor or other target cells.

In some embodiments, the Minn1 gene of the present invention delivered to the tumor cells of a subject using means other than viral gene transfer (e.g., via liposomes) is operably linked to nucleotide sequences which control expression of the Minn1 polypeptide, as discussed above.

In some embodiments, methods of gene therapy for the delivery the Minn1 gene to a subject involve parenteral administration. In some embodiments, systemic administration of the Minn1 gene is by intravenous or intra-arterial administration. In alternative embodiments, local administration is used. In one embodiment, local administration of the Minn1 gene is by surgical delivery, implant, or injection, or any other suitable method that restricts the distribution of the gene of the invention. In still further embodiments, an administration method is combined with catheter techniques and surgical operations.

As known to those in the art, the dosage levels of the agent for delivering the gene(s) of the invention vary depending on the age, sex and conditions of the subject, the route of administration, the number of administrations, and the type of the formulation, among other considerations. One skilled in the art is capable of determining the therapeutically effective amount appropriate any given circumstances. Usually, it is appropriate to administer a gene of the invention in an amount of 0.1-100 mg/adult body/day, although other concentrations are contemplated, as appropriate.

IX. Methods and Compositions for the Analysis of the Minn1 Gene, Transcript and Protein

The present invention provides the Minn1 gene, which has apoptosis inducing activity that is regulated by the Ras protein. It is shown herein that Minn1 is expressed in all normal tissues tested, and loss of Minn1 expression is observed in a majority of ovarian cancer cell lines tested.

It is contemplated that assessment of endogenous Minn1 expression will find use as a diagnostic tool in making the decision whether to treat a subject using a gene therapy protocol of the present invention. Prior to a gene therapy treatment comprising the Minn1 gene, it is contemplated that a biopsy sample taken from a subject's tumor will be analyzed for Minn1 expression or genomic status, as only tumors showing loss of the endogenous Minn1 expression are likely to benefit from Minn1 recombinant gene therapy. Furthermore, it is contemplated that subjects whose tumors display both loss of Minn1 expression and increased Ras activity are the most likely to benefit from gene therapy with the recombinant Minn1 gene.

The present invention provides compositions and methods for the assessment of endogenous Minn1 expression. In some embodiments, these methods and compositions are used alone or in combination, and include:

1) Northern blotting to detect endogenous Minn1 cDNA;

2) PCR analysis of genomic DNA for the detection of Minn1 gene deletion or rearrangements;

3) PCR analysis of cellular RNA to detect Minn1 transcripts;

4) Western immunoblotting using an anti-Minn1 antibody to detect Minn1 polypeptide;

5) ELISA assay to detect or quantitate Minn1 polypeptide;

6) Tissue typing arrays to expedite discovery of novel targets for cancer treatment; and

7) Test kits.

1) Northern Blotting to Detect Endogenous Minn1 cDNA

The present invention provides Northern blotting methods for the detection of endogenous Minn1 transcripts, as described in Example 5. In this Example, total cellular RNA was isolated using guanidinium isothiocyanate lysis followed by cesium chloride gradient purification. The RNA was resolved using denaturing agarose electrophoresis, blotted, and probed using a random-primed ³²P-dCTP labelled 813 bp PCR product corresponding to the full-length Minn1 cDNA.

In view of numerous alternative protocols known in the art for Northern blotting, it is not intended that the present invention be limited to the Northern blotting protocol provided in Example 5 or any other particular Northern blotting method. For example, in some embodiments, RNA is isolated from tissue samples using alternative methods (e.g., a commercial RNA isolation kit such as Qiagen RNeasy Total RNA Mini Kit, Catalog No. 74103).

Similarly, alternative probe synthesis and labelling techniques also find use with the present invention. For example, any probe having a minimum complementarity of 25 base pairs to the Minn1 cDNA will find use in the Northern blot methods of the present invention. Furthermore, it is contemplated that the nucleic acid comprising the probe will be generated by PCR, by restriction digest, or by synthetic oligonucleotide synthesis. Alternative nucleic acid probe labelling methods also find use with the present invention (e.g., labelling with ³³P radioisotope or non-radioactive labelling methods). In addition, alternative Northern blotting protocols and reagents suitable for use in the present invention are known in the art (See, e.g., Ausubel et al. (eds.), Current Protocols in Molecular Biology, Vol. 1, pages 4.9.1-4.9.16, John Wiley & Sons, Inc., New York [1994]).

2) PCR Analysis of Genomic DNA for the Detection of Minn1 Gene Deletions or Rearrangements

Analysis of genomic sequence databases using the Minn1 cDNA showed that the gene is located on human chromosome 3p21.3. Significantly, this region is frequently deleted or rearranged in human lung and ovarian carcinomas (Fullwood et al., Cancer Res., 59:4662-4667 [1999]), and is theorized to contain candidate tumor suppressor genes. It is shown herein that the Minn1 gene is a tumor suppressor gene that lies in this region and is deleted or rearranged in some cancers.

It is contemplated that Minn1 gene deletion or rearrangements will be detected by PCR analysis of genomic DNA isolated from tumor biopsy samples. In view of the numerous conditions known in the art for the analysis of genomic DNA by PCR, it is not intended that the present invention be limited to any particular method. Indeed, various combinations of PCR primers will find use in the present invention (e.g., where each set of primers flank or lie within the genomic region containing the Minn1 locus). It is not intended that the present invention be limited to the use of only one set of PCR primers flanking or lying within the Minn1 genomic locus, as numerous primer pairs will find use with the present invention. Suitable PCR primers result in the generation of a PCR product a minimum of 200 base pairs in length, more preferably 2000 base pairs in length, or more preferably longer than 2000 base pairs in length. The analysis of genomic DNA by PCR to detect genomic deletion or rearrangement is routine in the art, and is described in various sources, for example, Brkanac et al. (Am. J. Hum. Genet., 62(6):1500-1506 [1998]) and Valetto et al. (Electrophoresis 19(8-9):1385-1387 [1998]). PCR kits designed specifically for the amplification of long PCR products from eukaryotic genomes are available, and find use with the present invention (See, e.g., Roche Molecular Biochemicals, Expand 20 kb^{PLUS} and Long Template PCR Systems, Catalog Nos. 1811002 and 1681834, respectively). In addition to PCR methods, the isolation of genomic DNA is also routine in the art. Any suitable isolation method known in the art will find use with the present invention, including the use of genomic DNA isolation kits (e.g., Qiagen QIAamp Tissue Isolation Kit, Catalog No. 29304).

3) PCR Analysis of Cellular RNA to Detect Minn1 Transcripts

The present invention provides Northern blotting methods for the detection of endogenous Minn1 transcripts (See e.g., Example 5). However, in view of numerous alternative protocols known in the art for detection of gene transcripts, it is not intended that the present invention be limited to the Northern blotting protocol provided in Example 5 for the detection of Minn1 transcripts.

For example, in some embodiments, an mRNA transcript of the Minn1 gene is detected in total cellular RNA or polyA mRNA using reverse transcription polymerase chain reaction (RT-PCR). This technique, which incorporates a reverse transcriptase activity (i.e., an RNA-dependent DNA polymerase) as well as a DNA-dependent DNA polymerase activity, is known in the art, and is described in many sources (e.g., Mullis et al. (eds.), PCR—The Polymerase Chain Reaction, Chapter 24, “RT-PCR and Gene Expression,” Birkhauser Publishers, Cambridge, Mass. [1994]; and Ausubel et al. (eds.), Current Protocols in Molecular Biology, Section 15.4, “Enzymatic Amplification of RNA by PCR,” John Wiley & Sons, Inc., New York [1994]). In one embodiment, the reverse transcriptase and the DNA-dependent DNA polymerase activities are in separate enzymes. In a preferred embodiment, the reverse transcriptase and DNA-dependent DNA polymerase activities are encoded by the same enzyme. In a most preferred embodiment, the enzyme having both reverse transcriptase and DNA-dependent DNA polymerase activities is thermostable.

It is also not intended that the present invention be limited to the guanidinium isothiocyanate/cesium chloride RNA purification method described in Example 5. The art knows well alternative protocols for the isolation of total RNA or polyA mRNA. For example, commercial RNA isolation kits find use with the present invention (e.g., Qiagen RNeasy Total RNA Mini Kit, Catalog No. 74103).

4) Western Immunoblotting using an Anti-Minn1 Antibody to Detect Minn1 Polypeptide

The present invention provides monoclonal and polyclonal antibodies directed against Minn1 polypeptide. It is contemplated that the anti-Minn1 antibodies of the present invention will find use in Western immunoblotting to detect recombinant or endogenous Minn1 polypeptide, for example, endogenous Minn1 polypeptide in a tumor biopsy sample taken from a subject.

In view of the numerous conditions known in the art for the analysis of proteins by Western immunoblotting, it is not intended that the present invention be limited to any particular Western blotting method. For example, in some embodiments, tissue biopsy samples to be analyzed by Western immunoblotting using the anti-Minn1 antibody of the present invention are prepared by mechanical homogenization either manually (e.g., using a Dounce homogenizer) or by using a mechanical (i.e., electric) homogenizer. Before, during or after homogenization, tissue samples are suspended in a sample buffer suitable for loading directly onto an SDS-PAGE gel (e.g., Laemmli buffer). Following homogenization and addition of a suitable sample buffer, samples are heated, typically at 95° C. for 2 minutes, loaded and resolved on SDS-PAGE, blotted to a suitable substrate membrane (e.g., polyvinylidene difluoride [PVDF]), probed with an anti-Minn1 antibody of the present invention, followed by visualization with an appropriate secondary antibody.

The Examples provide descriptions of the use of Western blotting to assess Minn1 expression in cells. However, protocols and reagents for Western immunoblotting are well known to those in the art, and can be found in various sources (See, e.g., Ausubel et al. (eds.) (Current Protocols in Molecular Biology, Section 10.8, “Immunoblotting and Immunodetection,” John Wiley & Sons, Inc., New York [1994]; and Walker (ed.), The Protein Protocols Handbook, Part III, “Blotting and Detection Methods,” Humana Press, Totowa, N.J. [1996]). Thus, it is not intended that the present invention be limited to any particular method for performing Western blotting.

5) ELISA Assay to Detect and/or Quantitate Minn1 Polypeptide

As indicated above, the present invention provides monoclonal and polyclonal antibodies raised against Minn1 polypeptide. It is contemplated that the anti-Minn1 antibodies of the present invention find use in immunoassays such as enzyme-linked immunosorbent assays (ELISAs) to detect and/or quantitate recombinant or endogenous Minn1 polypeptide, for example, endogenous Minn1 polypeptide in a tumor biopsy sample taken from a subject.

Numerous ELISA methods are known in the art (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). Some ELISA formats known in the art which find use with the present invention include “direct ELISAs,” “indirect ELISAs” and “sandwich ELISAs.” However, in view of the numerous ELISA formats known in the art, it is not intended that the present invention be limited to any particular ELISA format.

Briefly, in some embodiments, these ELISA methods first immobilize a protein of interest that is in a sample (e.g., a protein extract from a tumor tissue biopsy) to a solid support (e.g., a microtiter plate well). In some embodiments, this immobilization is directly to the solid support, or via a suitable “capture antibody.” The anti-Minn1 antibody of the present invention finds use as a Minn1-specific capture antibody. Detection and quantitation of the immobilized antigen (i.e., the Minn1 polypeptide) is accomplished by the use of an antibody-enzyme conjugate detection antibody (i.e., the anti-Minn1 antibody of the present invention conjugated to a suitable enzyme) capable of binding to the immobilized antigen and producing a quantifiable signal. The amount of enzyme reaction product produced after the addition of a suitable enzyme substrate is directly proportional to the amount of antigen present in the sample. Enzymes commonly used in the ELISA detection step include horseradish peroxidase (HRPO), urease, alkaline phosphatase, glucoamylase and β-galactosidase. Methods for the preparation of suitable antibody-enzyme conjugates are also known to those skilled in the art. The end product of an ELISA is a signal, typically the development of color or fluorescence. Color development and fluorescence are read (i.e., quantitated) using a suitable spectrocolorimeter (i.e., a spectrophotometer) or spectrofluorometer, respectively. The amount of color or fluorescence is directly proportional to the amount of immobilized antigen.

6) Tissue Typing Arrays to Expedite Discovery of Novel Targets for Cancer Treatment

Tissue arrays provide means to screen a large number of samples in a short time using a high throughput system. During the development of the present invention, tissue arrays were produced and tested using tissue samples from controls and specimens suspected of expressing differing levels of Minn1 (e.g., loss of Minn1 expression). However, it is not intended that the present invention be limited to any particular method, system, or testing format for testing tissue samples. In some embodiments, microscope slides find use in these methods of the present invention to support the tissue samples. However, larger slides, plates and other formats to support the tissue samples find use with the present invention.

During the development of the present invention tissue arrays were obtained from the “Tissue Array Research Program” (“TARP”), a collaborative effort between the National Cancer Institute and the National Human Genome Research Institute (See, http://resresources.nci.nih.gov/tarp/). The tissue arrays used during the development of the present invention were provided as microarrays of 500 anonymized tumor and control tissue samples fixed onto glass slides (i.e., microscope slides). No clinical information regarding the samples was associated with the tissues used in the construction of these arrays. Upon receipt of the microarrays, immunohistochemical methods commonly used in the art, were employed to assess the level of Minn1 expression in the tissue samples. The antibodies used in these tests were those produced as described herein (e.g., Example 6), although it is not intended that the present invention be limited to any particular antibody or antibody preparation. Based on these results, appropriate therapy can be provided to the subjects tested.

Although immunohistochemical methods were used during the development of the present invention, any method that is suitable for tissue analysis finds use in the present invention. For example, methods including, but not limited to FISH, in situ hybridization, immunofluorescence (including confocal), radioimmunoassays, immunohistochemistry, and traditional histochemical staining methods all find use in the present invention.

7) Test Kits

The present invention further provides diagnostic kits useful for the rapid assessment of Minn1 genomic DNA, mRNA or polypeptide expression using either immunohistochemistry, Northern blotting, PCR analysis, Western blotting, or an enzyme-linked immunosorbent assay (ELISA), alone or in combination.

In some embodiments, kits designed to incorporate reagents for use in PCR methods include, but are not limited to, nucleic acid isolation reagents, PCR primers, PCR reaction buffer, deoxyribonucleotide triphosphates (dNTPs), thermostable reverse transcriptase, thermostable DNA-dependent DNA polymerase, thermostable enzyme having both reverse transcriptase and DNA-dependent DNA polymerase activities, and electrophoresis apparatus for visualization of the PCR products. In alternative embodiments, kits designed to facilitate Northern blotting include, but are not limited to, RNA purification reagents, electrophoresis and blotting apparatus, sample denaturation buffer, suitable blotting membrane (e.g., PVDF), nucleic acid suitable for use as a probe, and hybridization and wash buffers. In still further embodiments, kits designed to facilitate immunoassay protocols (i.e., Western immunoblots and ELISA assays) include, but are not limited to, tissue homogenizers, protein extraction buffers, protein PAGE sample buffers, electrophoresis and blotting apparatus, suitable primary and secondary antibodies, visualization reagents, microtiter plates, a suitable capture antibody, a suitable detection antibody (i.e., a suitable antibody-enzyme conjugate), suitable wash buffers, and a microtiter plate reader. In other embodiments, these kits further include any material(s) which make possible or facilitate the analysis of a sample, including, but not limited to, apparatus for sample collection, sample tubes, holders, trays, racks, dishes, plates, instructions to the kit user, solutions, buffers, and samples to be used for standardization, normalization, and/or control samples.

Experimental

In the experimental disclosure which follows, the following abbreviations apply: eq (equivalents); M (Molar); μM (micromolar); N (Normal); mol (moles); mmol (millimoles); μmol (micromoles); μmol (nanomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); U (units), mU (milliunits); min. (minutes); sec. (seconds); % (percent); kb (kilobase); bp (base pair); PCR (polymerase chain reaction); and BSA (bovine serum albumin).

Where manufacturers are indicated, the following abbreviations apply: Amersham or Amersham/Pharmacia (Amersham-Pharmacia Biotech, Inc., Piscataway, N.J.); BAbCO (BAbCO, Richmond, Calif.); Boehringer Mannheim (Boehringer Mannheim, Corp., Indianapolis, Ind.); Calbiochem (Calbiochem-Novabiochem, San Diego, Calif.); Clontech (Clontech, Palo Alto, Calif.); Gibco/BRL/Life Technologies (GIBCO BRL Life Technologies, Gaithersburg, Md.); Invitrogen (Invitrogen Corporation, Carlsbad, Calif.); Kodak (Eastman Kodak, Rochester, N.Y.); NEB (New England Biolabs, Beverly, Mass.); Promega (Promega Corp., Madison, Wis.); Viro Med (Viro Med Biosafety Lab, Camden, N.J.); Sigma (Sigma Chemical Co., St. Louis, Mo.); and Stratagene (Stratagene Inc., La Jolla, Calif.).

Restriction enzymes, other DNA modification enzymes and molecular biology reagents used in these Examples are readily available from numerous manufacturers, including, but not limited to, NEB, Boehringer Mannheim, Promega, Gibco/BRL and Stratagene.

The following Examples are provided in order to demonstrate and further illustrate certain preferred embodiments and aspects of the present invention and are not to be construed as limiting the scope thereof.

### EXAMPLE 1

### Tissue Culture and Transfections

N1H-3T3 cells (a mouse, contact inhibited embryonic cell line; ATCC CRL No. 1658) were propagated in Dulbecco's Modified Eagles Medium (DMEM) and 10% calf serum (Gibco-BRL). 293-T cells (a transformed human embryonal kidney cell line, ATCC CRL No. 1573) were grown in DMEM and 10% fetal calf serum (FCS). Cells were maintained using techniques common in the art (See e.g., Ausubel et al. (eds.), Current Protocols in Molecular Biology, Vol. 4, Section A.3F, “Techniques for Mammalian Cell Tissue Culture,” John Wiley & Sons, Inc., New York [1994]). Cells were transfected using the calcium phosphate precipitation technique as known in the art (Clark et al., Methods Enzymol., 255:395-412 [1995]). Stable transfections of expression vectors carrying the neo gene into NIH-3T3 cells used 200 ng of plasmid DNA per culture dish. Following transfection, stable transfectants were selected in 500 μg/ml G418 (Life Technologies).

### EXAMPLE 2

### Electronic Screening and Cloning

In an effort to identify novel gene products which are able to physically interact with the Ras protein, and are thus candidate Ras effectors, an electronic screen was undertaken to identify proteins containing the Ras-Association (RA) domain (Ponting and Benjamin, Trends Biochem. Sci., 21:422-425 [1996]), as exemplified by the RA domain of the mouse Nore1 Ras-effector protein, corresponding to amino acid residues 267-348 of that protein (Vavvas et al., Jour. Biol. Chem., 273(10):5439-5442 [1998]), having the sequence:

| | |
|---|---|
| ATTDKRTSFYLPLDAIKQLHISSTTTVSEVIQGLLK | (SEQ ID NO:3) |
| KFMVVDNPQKFALFKRIHKDGQVLFQKLSIADYP | |
| LYLRLLAGPDTDVLSFVLKENE | |

The expressed sequence tag (EST) database was searched using the National Center for Biotechnology Information (NCBI) search program “Advanced tBLASTn” using the amino acid sequence of the Nore1 RA domain (SEQ ID NO:3) as the search query. This query identified a 613 base pair human EST (GenBank Accession Number AA205984) encoding this motif. As this EST contained only a partial gene sequence, strategies were undertaken to identify the full length gene sequence.

Genetic material suitable for the identification and isolation of the full length cDNA was obtained from the IMAGE Consortium EST bank (IMAGE clone #632948). The full length cDNA was subcloned using a PCR strategy from the IMAGE consortium clone as a BamH1/EcoR1 PCR fragment using the following primers:

| | |
|---|---|
| 5′ primer: | |
| 5′-GACGGATCCATGGGCGAGGCGGAGGCGCC-3′ | (SEQ ID NO:4) |
| and | |
| 3′ primer: | |
| 5′-ACAGAATTCACCCAAGGGGGCAGGCG-3′ | (SEQ ID NO:5) |

The cDNA was sequenced and found to contain an 813 bp open reading frame (shown in FIG. 1 and SEQ ID NO:1). This open reading frame is predicted to encode a 270 amino acid protein (shown in FIG. 2 and SEQ ID NO:2).

This 270 amino acid sequence predicted by the cDNA open reading frame was used to search all GenBank sequences. This search demonstrated that the gene and protein of the present invention are novel, and the gene was named Minn1. This GenBank search revealed submissions of a similar, but not identical, 270 amino acid protein, differing at amino acid position 61 (Dammann et al., Nature Genetics 25:315-319 [2000]; and GenBank Accession Numbers AF040703, AF132676, AF061836 and NM_{—}007182). Thus, the present invention provides a novel protein.

### EXAMPLE 3

### In Vitro Ras/Minn1 Binding Assay

In this Example, experiments conducted to assess the ability of Ras and Minn1 to interact in vitro are described. These experiments used in vitro produced and purified MBP-Minn1 and MBP-Raf fusion proteins and purified Ras protein in a standard protein binding and co-precipitation assay. The MBP-Raf protein was included in the binding assays to serve as a positive control for GTP-dependent Ras binding.

A 638 base pair PCR product containing the Minn1 RA domain (spanning 211 amino acids, corresponding to amino acid positions 59-270) was generated as a BamH1/EcoR1 PCR fragment using an internal 5′ primer and a 3′ terminal primer (the same 3′ primer as was used in Example 2). These primers have the following sequences:

| | | |
|---|---|---|
| 5′ primer: | 5′-GACGGATCCGACCTTTCTCAAGCTGAGATTGAGC-3′ | (SEQ ID NO:6) |
| 3′ primer: | 5′-ACAGAATTGACCCAAGGGGGCAGGCG-3′ | (SEQ ID NO:5) |

The resulting PCR product encoding the Minn1 RA domain was cloned in-frame into a modified version of pMal (NEB) in which the orientation of the EcoR1/BamH1 sites in the multiple cloning site was reversed to BamH1/EcoR1. The construct encoding the MBP-Raf/RBD was made by subcloning a DNA fragment encoding amino acid residues 51-131 into pMal as described by Winkler et al. (J. Biol. Chem., 273:21578-21584 [1998]).

MBP-Minn1(RA) and MBP-Raf(RBD) fusion proteins were produced and purified by standard techniques known in the art (Clark et al, Jour. Biol. Chem., 272(34):20990-20993 [1997]). Briefly, recombinant proteins were produced in XL1-Blue Escherichia coli (Stratagene) and purified using maltose-conjugated sepharose beads. Following their purification to near homogeneity, concentrations of the fusion proteins were determined by SDS-PAGE followed by Coomassie Blue staining and comparison to known standards. Recombinant Ras protein was produced by inducing an H-Ras bacterial expression construct in bacteria followed by differential denaturation and dialysis, as known in the art (Campbell-Burk and Carpenter, Methods Enzymol., 255:3-13 [1995]).

In vitro binding assays contained 1 μg purified MBP-Minn1 (RA) bound to maltoheptaose beads and 10 μg of purified recombinant H-Ras, which had been preloaded with either GTP or GDP in a final volume of 500 μl RIPA buffer (150 mM NaCl, 1% Nonidet P-40 [NP-40], 0.5% sodium deoxycholate, 50 mM HEPES pH 7.4, 50 mM NaF, 2 μg/ml leupeptin, 2 μg/ml aprotinin and 1 μg/ml pepstatin A). The binding assays were performed at 4° C. for 2 hours in PBS containing 25 mM MgCl₂. Following this incubation, the reaction tube was spun at 12K rpm for 5 minutes in order to pellet the maltoheptose beads. The resulting pellet was washed four times in PBS containing 5 mM MgCl₂.

The washed and pelleted beads were resuspend in 40 μl of a standard 1× SDS-PAGE sample loading buffer containing 5% β-mercaptoethanol, then repelleted. From the resulting supernatant, 20 μl was loaded and resolved on a 4-20% Tris-glycine PAGE gel.

The proteins resolved in the PAGE were analyzed by Western immunoblotting using an anti-H-Ras monoclonal antibody (Viro Med). Briefly, proteins remaining in the binding reaction after the washes were resolved on 4-20% Tris-Glycine PAGE, transferred to a polyvinylidene difluoride (PVDF) membrane, probed with a 1:5000 dilution of the anti-Ras antibody, and then detected using an alkaline phosphatase conjugated secondary antibody and chemiluminescence detection. Nonspecific interactions between Ras and the MBP component of the fusion proteins was assessed by the amount of Ras captured using an equivalent amount of purified MBP protein.

As shown in FIG. 3, Ras protein was co-precipitated with the MBP-Raf protein (i.e., the positive control), indicating a physical interaction between Ras and MBP-Raf. Furthermore, as expected, this interaction was GTP-dependent. Interestingly, the Minn1 protein behaved in a similar fashion (i.e., Ras protein was also co-precipitated with the MBP-Minn1 protein), indicating a physical interaction between Ras and MBP-Minn1, which was also GTP-dependent. Alone, the MBP peptide showed no affinity for the Ras protein either in the presence or absence of GTP.

### EXAMPLE 4

### In Vivo Ras/Minn1 Binding Assay

In this Example, experiments conducted to assess the ability of H-Ras and Minn1 to interact in vivo are described. These experiments used a standard co-transfection/co-precipitation protocol with a FLAG-tagged-Minn1 expression vector and two HA-tagged H-Ras expression vectors, followed by immunoprecipitation with an anti-HA antibody and Western immunoblotting using an anti-FLAG primary antibody. The in vivo binding assay was conducted using 293-T cells, a transformed human embryonal kidney cell line.

The FLAG-Minn1 expression vector was constructed by subcloning a PCR product encoding the Minn1 coding sequence into pCDNAFLAG (Invitrogen), which is a version of pCDNA3 that was modified to add an upstream FLAG epitope tag to the amino terminal end of a cloned protein. To generate this PCR product, the same primers as described in Example 2 were used:

| | |
|---|---|
| 5′ primer: | |
| 5′-GACGGATCCATGGGCGAGCGGAGGCGCC-3′ | (SEQ ID NO:4) |
| and | |
| 3′ primer: | |
| 5′-ACAGAATTCACCCAAGGGGGCAGGCG-3′ | (SEQ ID NO:5) |

Two different HA-tagged forms of the HA-H-Ras protein were used in this assay. These were an expression vector encoding a wild-type HA-H-Ras fusion protein and an expression vector encoding an HA-H-Ras (G12V) gain-of-function mutation. The G12V mutation is known to be oncogenic, and results in elevated Ras signalling activity. This activated form of Ras typically shows greater than 70% association with GTP in vivo, while typically only 5% of wild-type Ras is bound to GTP. Thus, this mutant form of Ras is considered to be locked in an active conformation.

The HA-H-Ras(WT) expression vector was constructed by subcloning an H-Ras PCR product into pZipNeo SV(X)1HA, which is a modified form of pZipNeo SV(X)1 (Cepko et al., Cell 37:1053-1062 [1984]). This modified version of the plasmid has the internal EcoR1 site deleted and the cloning site modified from a single BamH1 site to a BamH1/HindIII/EcoR1 sequence downstream of an HA epitope (where the reading frame is GGA TTC). The following primers were used in this PCR reaction:

5′ primer:

(SEQ ID NO:7)

5′-GCGCGGATCCATGACAGAATACAAGCTTGTGG-3′
and
3′ primer:

(SEQ ID NO:8)

5′-GCGCGAATTCTCAGGAGAGCACACACTTGCAG-3′

The HA-H-Ras(G12V) gain-of-function gene was subcloned into pCGNHA, an HA-tagged expression vector described in Westwick et al. (Mol. Cell. Biol., 17:1324-1335 [1997]), to make the vector pCGNHA-H-Ras(G12V).

The in vivo binding assay was conducted by co-transfecting 100 ng of HA-H-Ras(WT) expression vector or 100 ng pCGNHA-H-Ras(G12V) expression vector with 10 μg pCDNAFLAG-Minn1 expression vector into 293-T cells. After 48 hours, the cells were lysed in EDTA-free RIPA buffer (described in Clark et al., J. Biol. Chem., 272:20990-20993 [1997]), immunoprecipitated with anti-HA antibody-conjugated sepharose beads (BAbCO), washed and subjected to Western immunoblotting using an anti-FLAG monoclonal primary antibody (M2 antibody, Sigma) and an alkaline phosphatase conjugated secondary antibody with an ECL chemiluminescence kit (Amersham).

The results of this in vivo binding assay are shown in FIG. 4. As indicated in the top portion of Panel A, the FLAG-tagged Minn1 preferentially associated with the activated HA-Ras(G12V) protein as compared to the HA-H-Ras(WT) protein. The expression of the FLAG tag alone (top portion of Panel B) confirms that there is no non-specific affinity between the FLAG tag and the Ras proteins. The Western blot in the lower portion of Panel A confirms adequate expression of the fusion proteins in the 293 cells. The result of this in vivo binding assay further confirms the observations made in the in vitro binding assay, where Minn1 preferentially bound to GTP-loaded Ras.

### EXAMPLE 5

### Northern Immunoblotting Analysis

In this Example, experiments conducted to analyze the expression pattern of the Minn1 gene by Northern blotting in a variety of human tissues as well as in normal and ovarian cancer cell lines are described.

FIG. 5 shows a multiple human tissue Northern blot (Clontech) probed with a Minn1 cDNA probe. The probe used in the Northern blot was made by random-primed ³²P-dCTP labelling of a 813 bp restriction fragment comprising the Minn1 coding region. Briefly, hybridization was performed in 500 mM NaPO₄H, 7% SDS, 1 mM EDTA pH 8.0, overnight at 65° C. The blot was then washed with two 30 minute washes of 40 mM NaPO₄H, 1% SDS, 1 mM EDTA pH 8.0, at 68° C., followed by autoradiography.

As indicated by FIG. 5, a single predominant transcript corresponding to the Minn1 gene was present in the RNA of each tissue tested. This Minn1 transcript is present to varying degrees, with some tissues showing stronger expression than other tissues.

FIG. 6 shows a Northern blot of total RNA prepared from normal and ovarian tumor cell lines and probed using the same Minn1 cDNA probe as used above. These cell lines included a non tumorigenic ovarian epithelial cell line IOSE-120, as well as ovarian tumor cell lines OVCAR-3, OVCAR429, A364, A547, OVT2, A2780, UC1101, UC1107 and CaOV3. Briefly, total RNA was prepared from these cell lines using guanidinium isothiocyanate lysis followed by cesium chloride gradient purification. Samples containing 10 μg of the total RNA from each of the cell lines were resolved on a 0.8% denaturing agarose-formaldehyde gel using standard techniques. Following resolution, the gel was blotted onto nylon membrane. Probe hybridization was performed in 500 mM Na₂HPO₄, 7% SDS, 1 mM EDTA pH 8.0, overnight at 65° C. The blot was then washed twice with 40 mM NaPO₄H, 1% SDS, 1 mM EDTA pH 8.0, at 68° C. for 30 minutes each wash, and followed by autoradiography.

As indicated by FIG. 6, the non-transformed ovarian cell line IOSE-120 shows a single RNA species corresponding to the Minn1 gene, while the majority of the ovarian cancer cell lines (6 of 9) do not show any Minn1 expression. Thus, the present invention provides methods and compositions suitable for the assessment of cancerous cells.

### EXAMPLE 6

### Western Blotting of Human Tumor Cell Lines

In this Example, Western blots prepared using various human tumor cell lines are described The antibody preparation used in these experiments contained polyclonal antibodies directed against amino acids 151-171 of the internal peptide sequence RAREVIEALLRKFLVVDDPRK (SEQ ID NO:9). More particularly, this Example describes the use of the antibody preparation to examine the expression of Minn1 in human ovarian, lung and breast tumor cells. This is significant in that it provides proof that Minn1 is a Ras oncoprotein effector.

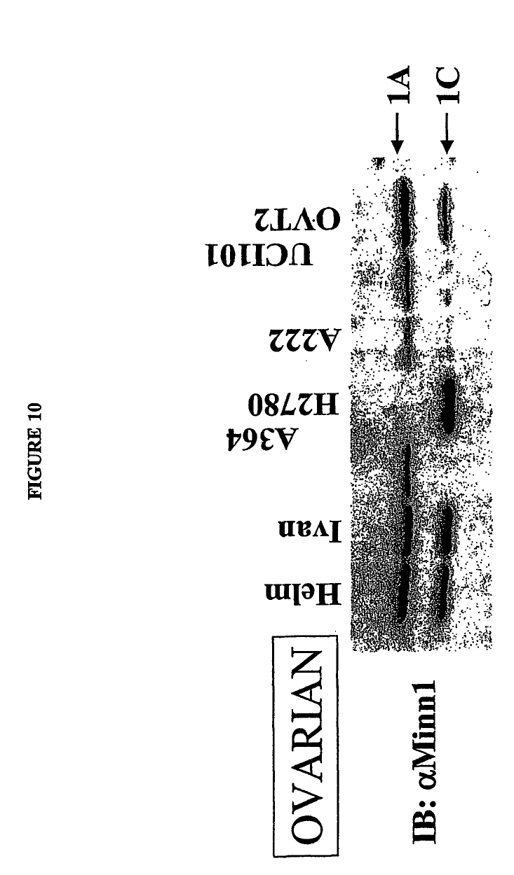

Using methods well-known in the art, lysates from seven (7) human ovarian tumor cell lines were examined, as well as non-transformed human lung epithelial cells, epithelial lung tumor cells, and breast tumor cells. As indicated in FIG. 10, Minn1 protein can be expressed as two different isoforms (Minn1A and Minn1C), which is compatible with the exon structure of the gene. No complete loss of expression was observed in these ovarian samples.

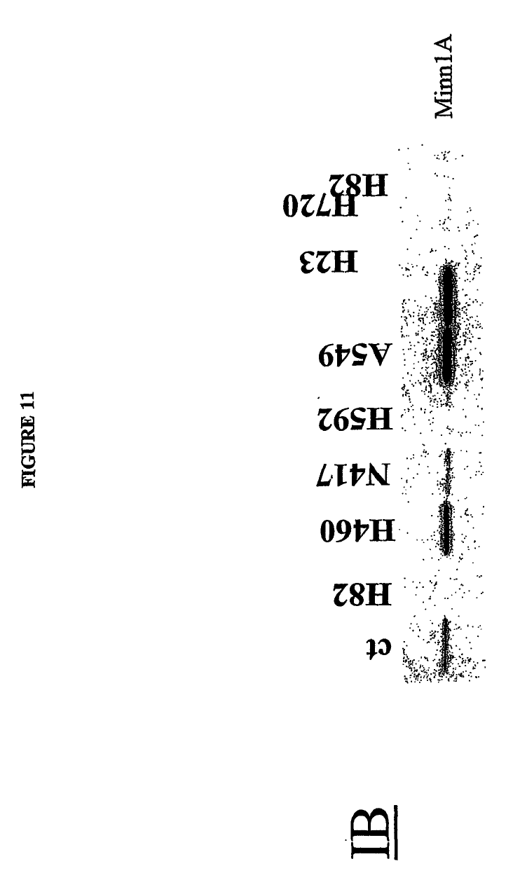

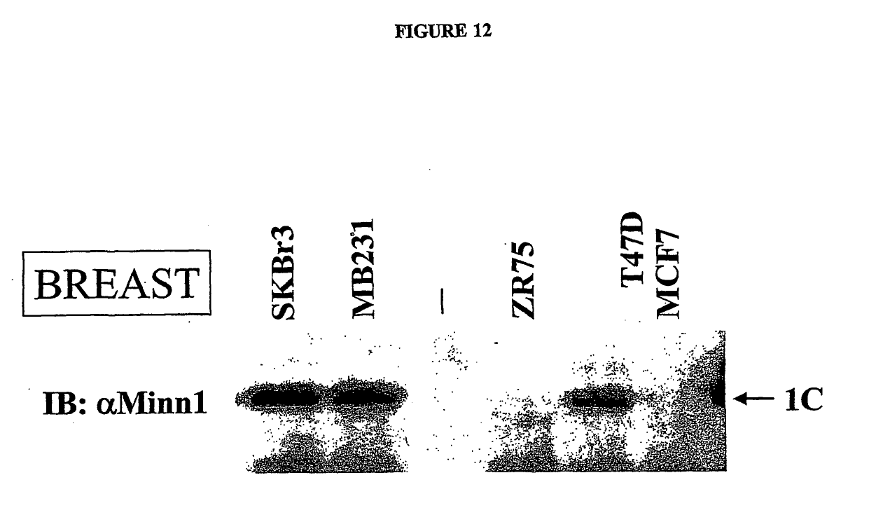

As indicated in FIG. 11, non-transformed human lung epithelial cells (ct) express only the 1A form of Minn1 and this is absent or severely reduced in 4 out of 7 of the epithelial lung tumor cell lines examined. In contrast, as indicated in FIG. 12, human breast tumor cells express only the 1C isoform of Minn1 and this is absent in ⅖ of the tumor cell lines examined.

Thus, the data obtained in these experiments indicate that Minn1 is frequently down-regulated in human tumor cells, compatible with a role as a tumor suppressor. The tissue specific isoform expression is intriguing but is of unknown significance.

### EXAMPLE 7

### Interaction of Endogenous Ras in Minn1

This Example describes experiments conducted to determine whether Minn1 interacts with Ras in vivo. Minn1 has an RA (Ras association) domain which appears to interact directly with the Ras oncoprotein in experimental systems. However, to confirm that this interaction is physiological, it was necessary to show that endogenous Minn1 can interact with the endogenous Ras oncoprotein.

In these experiments, two human tumor cell lines, EJ bladder carcinoma and MiaPaCa pancreatic carcinoma were examined. These cell lines express activated H-Ras and K-Ras, respectively. Both also express Minn1, which can be detected as two isoforms, A and C.

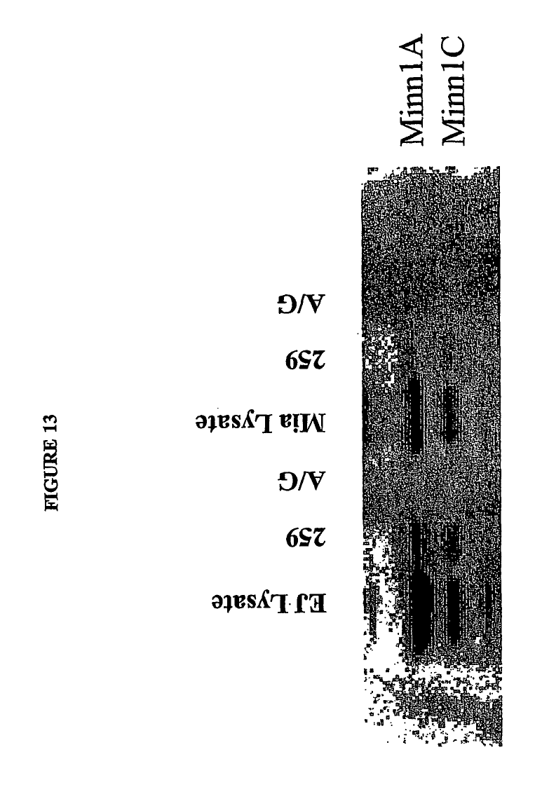

In these experiments, cell lysates were immunoprecipitated with 259 pan Ras antibody (Santa Cruz Biotechnology) using methods well-known in the art. The samples were then examined by Western blot using Minn1 antibody described in Example 6 and methods known in the art. In FIG. 13, lane A is the positive control, showing a lysate sample with Minn1 isoforms A and C; lane B shows the lysate immunoprecipitated with pan Ras which also shows Minn1 A and C coming down with the Ras; lane C is the negative control showing the lysate precipitated with A/G beads alone. As Minn1 bands can be seen in the Ras immunoprecipitate but not in the A/G beads precipitate, these data support the conclusion that endogenous Minn1 can associate with endogenous Ras in vivo.

### EXAMPLE 8

### Tissue Array Testing

In this Example, methods involving the use of tissue arrays to assess Minn1 expression are described.

Tissue arrays were produced and tested using tissue samples from controls and specimens suspected of expressing differing levels of Minn1 (e.g., loss of Minn1 expression). The tissue arrays were obtained from the “Tissue Array Research Program” (“TARP”), a collaborative effort between the National Cancer Institute and the National Human Genome Research Institute (for more information, See, http://resresources.nci.nih.gov/tarp/). These tissue arrays were provided as microarrays of 500 anonymized tumor and control tissue samples fixed onto glass slides (i.e., microscope slides). No clinical information regarding the samples was associated with the tissues used in the construction of these arrays. Upon receipt of the microarrays, immunohistochemical methods commonly used in the art, were employed to assess the level of Minn1 expression in the tissue samples. Data were analyzed using software and manual data analysis methods.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, genetics, cancer biology or related fields are intended to be within the scope of the following claims.

What is claimed is:

1. An isolated nucleic acid encoding the polypeptide set forth in SEQ ID NO:2.

2. The nucleic acid of claim 1, where in said nucleic acid comprises the nucleotide sequence set forth in SEQ ID NO:1.

3. An isolated polypeptide having the amino acid sequence set forth in SEQ ID NO:2.

4. A composition comprising the nucleic acid of claim 1.

5. A recombinant vector comprising the nucleic acid of claim 1.

6. The recombinant vector of claim 5, wherein said vector is an expression vector.

7. A host cell comprising the recombinant vector of claim 5, wherein said host cell is selected from the group consisting of prokaryotic host cells and eukaryotic host cells.

8. An antibody directed against at least a portion of the amino acid sequence of SEQ ID NO:2, wherein said antibody is selected from the group consisting of monoclonal antibodies and polyclonal antibodies.

9. A composition comprising the antibody of claim 8.

10. A method for treating a subject, comprising the steps of:
a) providing:
i) a subject,
ii) the recombinant vector of claim 5,
iii) a target within said subject,
iv) a means of delivery of said vector to said target within said subject; and
b) delivering said vector to said target within said subject using said means of delivery.

11. The method of claim 10, wherein said subject is a human.

12. The method of claim 10, wherein said target is a solid tumor.

13. The method of claim 12, wherein cells comprising said solid tumor comprise at least one mutation in at least one Ras-family gene, wherein said mutation results in increased Ras signalling activity.

14. The method of claim 12, wherein cells comprising said solid tumor comprise reduced levels of a Minn1 product relative to non-tumor tissue of like origin, wherein said Minn1 product is selected from the group consisting of Minn1 transcript and Minn1 polypeptide.

15. The method of claim 12, wherein said solid tumor comprises cells containing at least one mutation in at least one Ras-family gene, wherein said mutation results in increased Ras signalling activity, and wherein cells comprising said solid tumor comprise reduced levels of a Minn1 product selected from the group consisting of Minn1 transcript and Minn1 protein.

16. The method of claim 12, wherein said solid tumor is an ovarian tumor.

17. The method of claim 10, wherein said means of delivery is selected from the group consisting of liposome-DNA complexes and recombinant viruses.

18. The method of claim 17, wherein said recombinant virus comprises operably linked recombinant nucleotide sequences comprising a suitable promoter sequence and viral sequences, wherein said viral sequences are selected from the group consisting of adenovirus sequences, adeno-associated virus sequences, retrovirus sequences, herpes virus sequences, vaccinia virus sequences and Moloney virus sequences.

19. The method of claim 10, wherein said means of delivery is selected from local delivery and systemic delivery, and wherein local delivery is selected from the group comprising surgical delivery, implantation, and injection.

20. A method for detecting a Minn1 polypeptide in a sample, comprising:
a) providing:
i) a sample,
ii) an antibody directed against a Minn1 polypeptide;
b) contacting said sample with said antibody under conditions such that said antibody specifically binds to said Minn1 polypeptide in said sample to form an antigen-antibody complex; and
c) detecting said antigen-antibody complex.

21. The method of claim 20, wherein said antibody is directed against an isoform of Minn1.

22. The method of claim 20, wherein said sample is from a human subject.

23. The method of claim 22, wherein said sample comprises tumor tissue.

24. The method of claim 20, wherein said method comprises a Western immunoblot assay.

25. The method of claim 20, wherein said method comprises an enzyme-linked immunosorbent assay.

26. The method of claim 25, wherein said enzyme-linked immunosorbent assay is selected from the group consisting of direct enzyme-linked immunosorbent assays, indirect enzyme-linked immunosorbent assays, direct sandwich enzyme-linked immunosorbent assays, indirect sandwich enzyme-linked immunosorbent assays, and competitive enzyme-linked immunosorbent assays.

27. A method for detecting a Minn1 transcript in a sample, comprising
a) providing:
i) a sample, wherein said sample comprises RNA selected from the group consisting of total cellular RNA and polyA RNA,
ii) a nucleic acid probe having complementarity to at least a portion of the nucleotide sequence of SEQ ID NO:1,
iii) a means of detecting a hybridization complex comprising said probe;
b) combining said nucleic acid probe and said sample under conditions suitable for the formation of a hybridization complex between said probe and said Minn1 transcript, and
c) detecting said hybridization complex.

28. The method of claim 27, wherein said sample is from a human subject.

29. The method of claim 28, wherein said sample comprises tumor tissue.

30. The method of claim 27, wherein said method comprises a Northern blot.

31. A method for detecting a Minn1 transcript in a sample, comprising:
a) providing:
i) a sample, wherein said sample comprises RNA selected from the group consisting of total cellular RNA and polyA RNA;
ii) a reverse transcriptase RNA-dependent DNA polymerase activity;
iii) PCR primers having complementarity to the nucleotide sequence of SEQ ID NO:1;
iv) a DNA-dependent DNA polymerase activity;
v) PCR amplification reagents;
b) reverse transcribing said RNA in said sample to form a double stranded DNA template;
c) annealing said primers to said template;
d) extending said primers with reiterated DNA synthesis under conditions such that said DNA template is amplified to produce an amplified product; and
e) detecting said amplified product.

32. The method of claim 31, wherein said sample is from a human subject.

33. The method of claim 32, wherein said sample comprises tumor tissue.

34. The method of claim 31, wherein said reverse transcriptase RNA-dependent DNA polymerase activity DNA-dependent DNA polymerase activity are thermostable.

35. The method of claim 31, wherein said reverse transcriptase RNA-dependent DNA polymerase activity DNA-dependent DNA polymerase activity are comprised in the same protein.

36. A method for detecting deletion mutations in a Minn1 genomic locus, comprising:
a) providing:
i) a first sample, wherein said first sample comprises genomic DNA from tumor tissue;
ii) a second sample, wherein said second sample comprises genomic DNA from a non-tumorigenic tissue;
iii) PCR primers;
iv) a DNA-dependent DNA polymerase;
v) PCR amplification reagents;
b) annealing said primers to said genomic DNA of said first and said second samples;
c) extending said primers with reiterated DNA synthesis under conditions to produce a first amplified product from said first sample and a second amplified product from said second sample;
d) detecting said first and second amplified products; and
e) comparing said first and second amplified products.

37. The method of claim 36, wherein said first and second samples are from at least one human subject.

38. A method for detecting a Minn1 polypeptide in an array of tissue samples, comprising:
a) providing:
i) a tissue array comprising at least two tissue samples,
ii) an antibody directed against a Minn1 polypeptide;
b) contacting said tissue samples with said antibody under conditions such that said antibody specifically binds to said Minn1 polypeptide in said tissue samples to form an antigen-antibody complex; and
c) detecting said antigen-antibody complex.

39. The method of claim 38, wherein at least one of said tissue samples is from a human subject.

40. The method of claim 38, wherein said sample comprises tumor tissue.

41. The method of claim 38, wherein said method comprises an immunohistochemical testing assay.

42. The method of claim 38, wherein said tissue array comprises more than 100 tissue samples.

43. The method of claim 38, wherein said tissue array comprises tissue samples from normal and tumor tissues.

44. The method of claim 38, further comprising the step of determining the cell type in said tissue sample that exhibits said antigen-antibody complex.


SEQUENCE LISTING (10 sequences)

SEQ ID NO: 1 - DNA, 813 bp, Homo sapiens
atgggcgaggcggaggcgccttctttcgaaatgacctggagcagcacgacgagcagtggctactgcagccaagaggactcggactcggagctcgagcagtacttcaccgcgcgaacctcgctagctcgcaggccgcgccgggaccaggacgagcctgtggagtgggagacacctgacctttttcaagctgagattgagcagaagatcaaggagtacaatgcccagatcaacagcaacctcttcatgagcttgaacaaggacggttcttacacaggcttcatcaaggttcagctgaagctggtgcgccctgtctctgtgccctccagcaagaagccaccctccttgcaggatgcccggcggggcccaggacggggcacaagtgtcaggcgccgcacttccttttacctgcccaaggatgctgtcaagcacctgcatgtgctgtcacgcacaagggcacgtgaagtcattgaggccctgctgcgaaagttcttggtggtggatgacccccgcaagtttgcactctttgagcgcgctgagcgtcacggccaagtgtacttgcggaagctgttggatgatgagcagcccctgcggctgcggctcctggcagggcccagtgacaaggccctgagctttgtcctgaaggaaaatgactctggggaggtgaactgggacgccttcagcatgcctgaactacataacttcctacgtatcctgcagcgggaggaggaggagcacctccgccagatcctgcagaagtactcctattgccgccagaagatccaagaggccctgcacgcctgcccccttgggtga

SEQ ID NO: 2 - protein, 270 aa, Homo sapiens
MGEAEAPSFEMTWSSTTSSGYCSQEDSDSELEQYFTARTSLARRPRRDQDEPVEWETPDLFQAEIEQKIKEYNAQINSNLFMSLNKDGSYTGFIKVQLKLVRPVSVPSSKKPPSLQDARRGPGRGTSVRRRTSFYLPKDAVKHLHVLSRTRAREVIEALLRKFLVVDDPRKFALFERAERHGQVYLRKLLDDEQPLRLRLLAGPSDKALSFVLKENDSGEVNWDAFSMPELHNFLRILQREEEEHLRQILQKYSYCRQKIQEALHACPLG

SEQ ID NO: 3 - protein, 92 aa, Mus musculus
ATTDKRTSFYLPLDAIKQLHISSTTTVSEVIQGLLKKFMVVDNPQKFALFKRIHKDGQVLFQKLSIADYPLYLRLLAGPDTDVLSFVLKENE

SEQ ID NO: 4 - DNA, 29 bp, Artificial
  Synthetic
gacggatccatgggcgaggcggaggcgcc

SEQ ID NO: 5 - DNA, 26 bp, Artificial
  Synthetic
acagaattcacccaagggggcaggcg

SEQ ID NO: 6 - DNA, 34 bp, Artificial
  Synthetic
gacggatccgacctttctcaagctgagattgagc

SEQ ID NO: 7 - DNA, 32 bp, Artificial
  Synthetic
gcgcggatccatgacagaatacaagcttgtgg

SEQ ID NO: 8 - DNA, 32 bp, Artificial
  Synthetic
gcgcgaattctcaggagagcacacacttgcag

SEQ ID NO: 9 - protein, 21 aa, Homo sapiens
RAREVIEALLRKFLVVDDPRK

SEQ ID NO: 10 - protein, 15 aa, Homo sapiens
QEDSDSELEQYFTAR